(19) United States
(12) Patent Application Publication
(10) Pub. No.: US 2022/0330823 A1
(43) Pub. Date: Oct. 20, 2022

(54) SYSTEMS AND DEVICES FOR TELEMETRY MONITORING MANAGEMENT

(71) Applicant: GE Precision Healthcare LLC, Wauwatosa, WI (US)

(72) Inventor: Brian Janssen, Brookfield, WI (US)

(21) Appl. No.: 17/855,407

(22) Filed: Jun. 30, 2022

Related U.S. Application Data
(63) Continuation-in-part of application No. 16/532,366, filed on Aug. 5, 2019.

(51) Int. Cl. A61B 5/00; G16H 10/60

(52) U.S. Cl. CPC A61B 5/0022; A61B 5/743; A61B 5/746; A61B 5/7264; A61B 5/7282; G16H 10/60

(57) ABSTRACT

A system for telemetry management comprising a plurality of telemetry monitoring sensors and a telemetry management system. The telemetry monitoring sensors send physiological information to the telemetry management system over a wireless network. The telemetry management system includes a processor which obtains physiological information from the telemetry monitoring sensors and classifies patients based on the physiological information. The telemetry management system also includes a display which displays information corresponding to the patients that is arranged based on the classifications.

### PRIORITY

This Application is a continuation-in-part of U.S. patent application Ser. No. 16/532,366, filed on Aug. 5, 2019, which is incorporated herein by reference in its entirety.

### FIELD

Embodiments of the subject matter disclosed herein relate to medical information systems, and more particularly, to medical telemetry patient monitoring.

### BACKGROUND

In hospitals and other patient treatment facilities, clinicians often desire to monitor multiple physiological characteristics for multiple patients. For some patient conditions, it may be desirable to provide uninterrupted monitoring for a duration. Additionally, ambulation of the patient is often desirable in order to stimulate patient recovery. To provide uninterrupted monitoring with patient ambulation, clinicians may order telemetry monitoring of patients, where portable sensors are coupled to the patients and the sensors are configured to measure patient vital signs and other patient health parameters. The sensors may transmit patient health data to one or more computing systems located remotely from the patient, where the data may be continuously observed by facility staff for indicators of degradation of the condition of the patients.

### BRIEF DESCRIPTION

According to an aspect of the disclosure, a system for telemetry monitoring may include: a plurality of telemetry monitoring sensors, each telemetry monitoring sensor including: a battery; a radio antenna configured to communicate over a wireless network; at least one sensor configured to measure a physiological parameter; a memory configured to store instructions; and a controller configured to execute the instructions to control the radio antenna to send electronic signals over the wireless network that include real time or near real time machine data obtained from the at least one sensor, the real time or near real time machine data including physiological information; and a telemetry management system including: a display device; a communication interface configured to communicate over the wireless network; a memory configured to store instructions; and at least one processor configured to execute the instructions to: obtain patient information for a plurality of patients; receive, through the communication interface, the physiological information from the plurality of telemetry monitoring sensors, the physiological information respectively corresponding to the plurality of patients; obtain secondary information including patient monitoring standards and protocols; classify each patient of the plurality of patients based on one or more of the patient information, the physiological information, and the secondary information; and control the display device to display information corresponding to the classifications the plurality of patients, and wherein the classifications include addition, discontinuation, and compliant.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1A is a diagram of a system for telemetry monitoring according to an example embodiment.

FIG. 1B is a block diagram illustrating a medical information system including a telemetry management system according to an exemplary embodiment.

FIG. 2 is a flow chart illustrating a method for managing telemetry protocol via a telemetry management system, according to an exemplary embodiment.

FIG. 3 is a flow chart illustrating a method for determining a telemetry monitoring status for each patient in a patient index via a telemetry management system, according to an exemplary embodiment.

FIGS. 4A-4B show a flow chart illustrating a method for determining a score for each patient in a patient index based on patient information and telemetry monitoring status via a telemetry management system, according to an exemplary embodiment.

FIG. 5 is a block diagram illustrating acquisition of a patient index including a list of patients and patient information by the telemetry management system of FIG. 1B, according to an exemplary embodiment.

FIG. 6 is a block diagram illustrating forming a stratified patient list based on a score of each patient via the telemetry management system of FIG. 1B and displaying the stratified patient list at a display device of the telemetry management system, according to an exemplary embodiment.

FIG. 7 is a block diagram illustrating forming a stratified patient list based on a score of each patient via the telemetry management system of FIG. 1B and displaying the stratified patient list at a display device of the telemetry management system, according to another exemplary embodiment.

FIGS. 8A-8C show a timeline of patient scores over time for two example patients being monitored with the telemetry management system of FIG. 1B, while FIG. 8D shows a timeline of patient score over time for a third example patient being monitored with the telemetry management system of FIG. 1B, according to an exemplary embodiment.

FIG. 9 is a flowchart of a process of telemetry monitoring management according to an example.

FIGS. 10A and 10B show an example of an output of the telemetry management system 100.

FIG. 11 shows another example of an output the telemetry management system 100.

FIG. 12 is a flowchart of a process of telemetry monitoring management according to another example.

### DETAILED DESCRIPTION

The following description relates to various embodiments for a telemetry management system. Telemetry patient monitoring may include monitoring patients remotely (e.g., wirelessly) via one or more diagnostic sensors, such as electrocardiogram (ECG) sensors, peripheral capillary oxygen saturation (SpO2) sensors, respiration rate sensors, temperature sensors, blood pressure sensors, etc. The sensors may be small, portable devices (whether as multiple separate sensors or as ab integrated acquisition device) coupled to the patient in order to enable the patient to ambulate (e.g., walk) remotely relative to a designated hospital bed or treatment room while maintaining monitoring of the condition of the patient (e.g., heart rhythm, oxygenation, and other patient vitals). For example, ambulation of the patient may be desirable for resolution of various medical conditions for which the patient is being treated (e.g., chest pain, cardiovascular syncope, etc.). A care provider (e.g., nurse, clinician, etc.) may view an output of the sensors at a remote location or handheld device throughout the duration that the sensors are coupled to the patient.

However, telemetry monitoring of patients is often overprescribed. For example, clinicians may order telemetry monitoring of patients for conditions that are outside of a recommended telemetry monitoring protocol for the hospital or facility as a precaution. Hospitals and other care facilities utilizing patient telemetry monitoring may routinely treat a large number of patients (e.g., 300 patients, 400 patients, etc.). Because each patient coupled to the sensors for telemetry monitoring is monitored remotely by a care provider, regularly monitoring patients via telemetry monitoring for non-recommended conditions may increase a cost of treatment of the patients and a difficulty of managing treatment. Each patient is coupled to a separate set of sensors, which may increase a total number of sensors utilized for telemetry monitoring of the patient population of the hospital. Telemetry monitoring, when used outside a recommended telemetry monitoring protocol, may decrease patient satisfaction/comfort due to the number of sensors that may be coupled to the patient. Further, electrodes and/or wires of the sensors may be replaced at regular intervals and may increase a maintenance cost associated with telemetry monitoring. As the number of patients being monitored via telemetry monitoring increases, a number of care providers monitoring the output of the sensors coupled to the patients also increases, which may result in an increased care provider training cost and/or operational cost of the facility. Additionally, a facility may have a finite amount of medical equipment (e.g., beds, telemetry monitoring devices, etc.) allocated toward providing telemetry monitoring to patients. As the number of patients being monitored via telemetry monitoring increases, the amount of equipment available for other patients (e.g., patients not yet being monitored via telemetry monitoring) decreases. Overprescribing telemetry monitoring may result in less medical equipment available for patients that are not yet undergoing telemetry monitoring. Further still, telemetry monitoring may result in excess alarms, which may lead to care provider alarm fatigue. Additionally, patients undergoing telemetry monitoring for relatively low risk conditions may distract from care delivery for patients having higher risk conditions, which may reduce efficiency and delay a response time of care providers.

The telemetry management system described herein graphically provides a stratified patient list showing patient names, rank and/or score, and alerts in order to reduce a complexity and cost associated with telemetry patient monitoring. The patient names are stratified by the telemetry management system based on score in order to graphically indicate a priority of patients that may be removed from telemetry monitoring. For example, patients with a higher rank or score may be candidates for telemetry monitoring cessation while patients with a lower rank or score may be candidates for continued telemetry monitoring. Additionally, the stratified patient list may include alerts in order to indicate a recommended course of action (e.g., cessation of telemetry monitoring, initiation of telemetry monitoring, increasing a duration of telemetry monitoring, etc.) for each patient based on patient telemetry monitoring status and patient information acquired from one or more databases. Each patient may be assigned a score upon admission of the patient to the medical facility, and the score may increase or decrease based on the patient telemetry monitoring status (e.g., duration of telemetry monitoring) and/or relevant patient information. For example, if a change in the patient information occurs that indicates potential health issues, such as a lab result indicating abnormal troponin levels, medication changes, dysrhythmia, high or low heart rate, or low oxygenation, the telemetry management system may reduce the patient score in order to decrease the rank of the patient within the stratified patient list. Additionally, the telemetry management system may provide an alert for the care provider(s) to increase the duration of telemetry monitoring of the patient.

The stratified patient list is viewable via a display device of the telemetry management system and/or on a display device of an alert notification system. In some embodiments, the stratified patient list may be viewable via a plurality of computing systems electronically coupled to the telemetry management system via a network. Thus, care providers may have access to the stratified patient list and may view patient alerts from virtually any allowed location within the medical facility, and even off-site locations in some embodiments.

In order to accurately diagnose a patient condition and/or determine a duration of patient telemetry monitoring, a care provider may order one or more lab tests, where a patient specimen is sent to an on-site or off-site laboratory and lab service providers at the laboratory analyze the specimen and send the results of the analysis back to the care provider. Further, patient medication status may be determined, where the patient medication status may include administration of one or more new medications, which may be dispensed by a pharmacist at an on-site or an off-site pharmacy, as well as determination of any medications administered to the patient prior to admission to the medical facility (e.g., medications the patient was already taking at time of admission). To ensure that patients are not prematurely removed from telemetry monitoring, the telemetry management system may adjust the score of patients in response to current and new and/or updated patient information (e.g., lab results, pharmacy orders, current medications, diagnosis) acquired by the telemetry management system. For example, lab results acquired by the telemetry management system may indicate that telemetry monitoring should be started, continued, and/or prolonged (e.g., due to an increased likelihood of deterioration of a patient's condition or lack of improvement in the patient condition). As a result, the telemetry management system may reduce the score of the patient and set an alert to notify a clinician to initiate telemetry monitoring of the patient (e.g., if the patient is not currently being monitored via telemetry monitoring) or increase a duration of telemetry monitoring of the patient (e.g., if the patient is currently being monitored via telemetry monitoring). In this way, a lab result that is outside of a normal range may justify continued telemetry monitoring based on relevant protocols or guidelines.

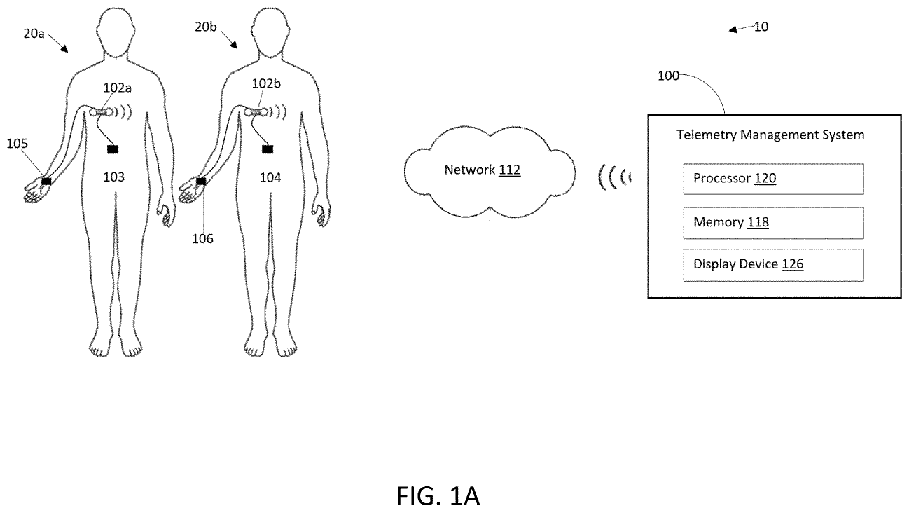

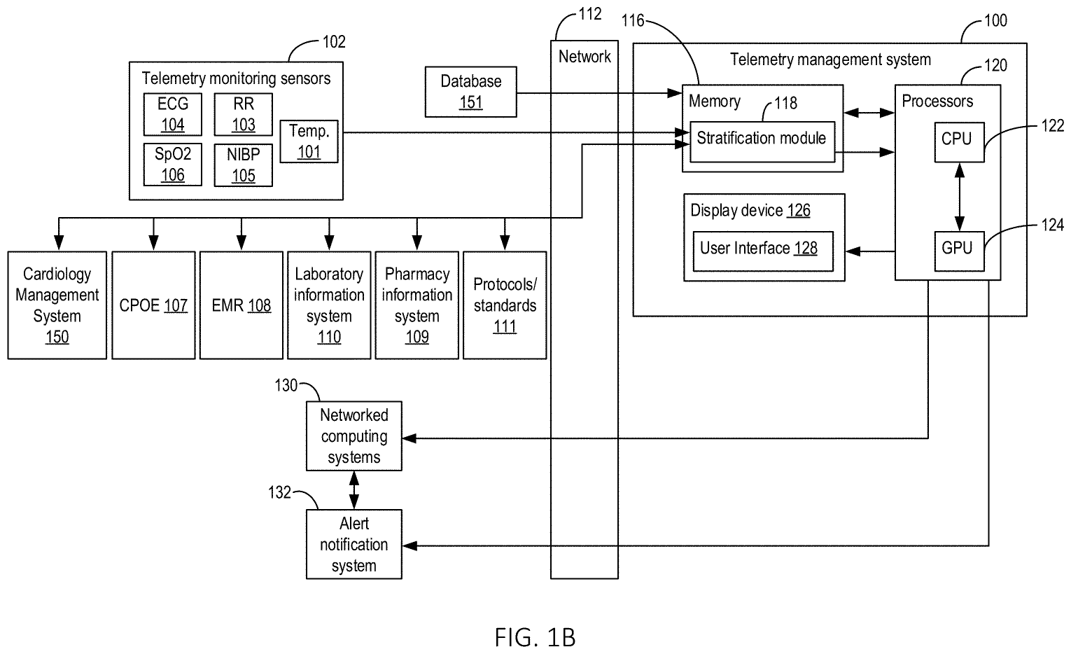

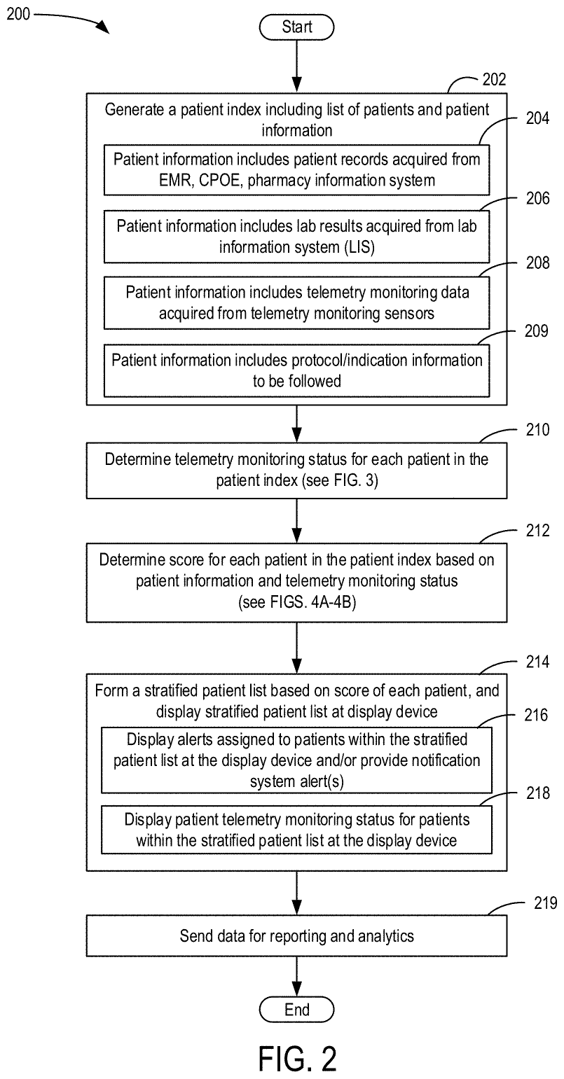

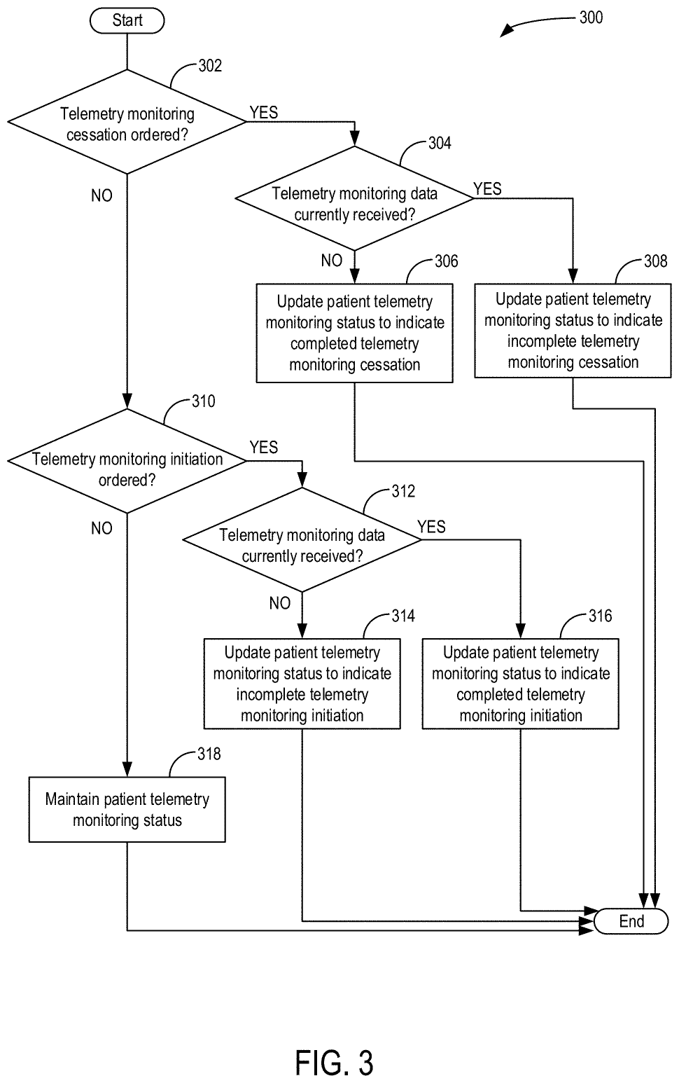

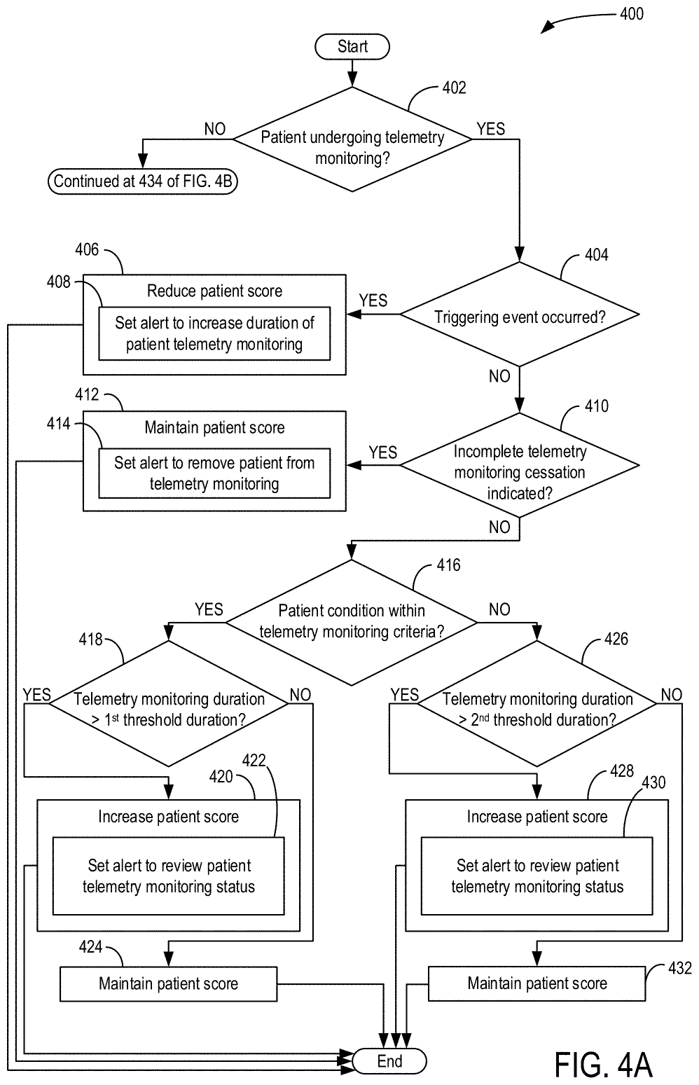

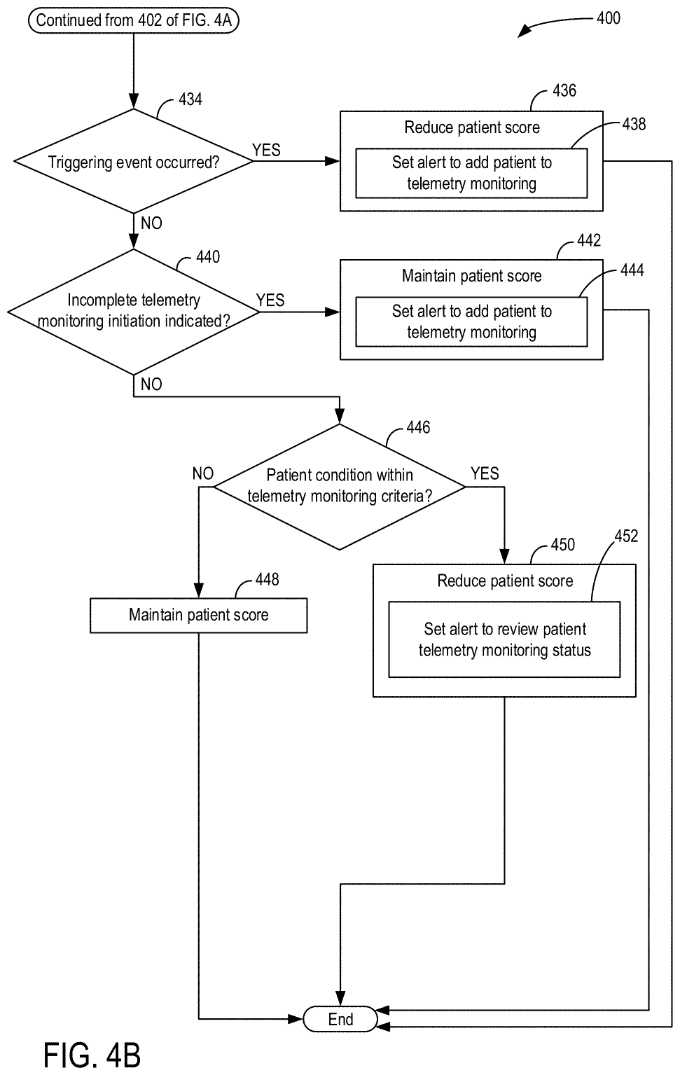

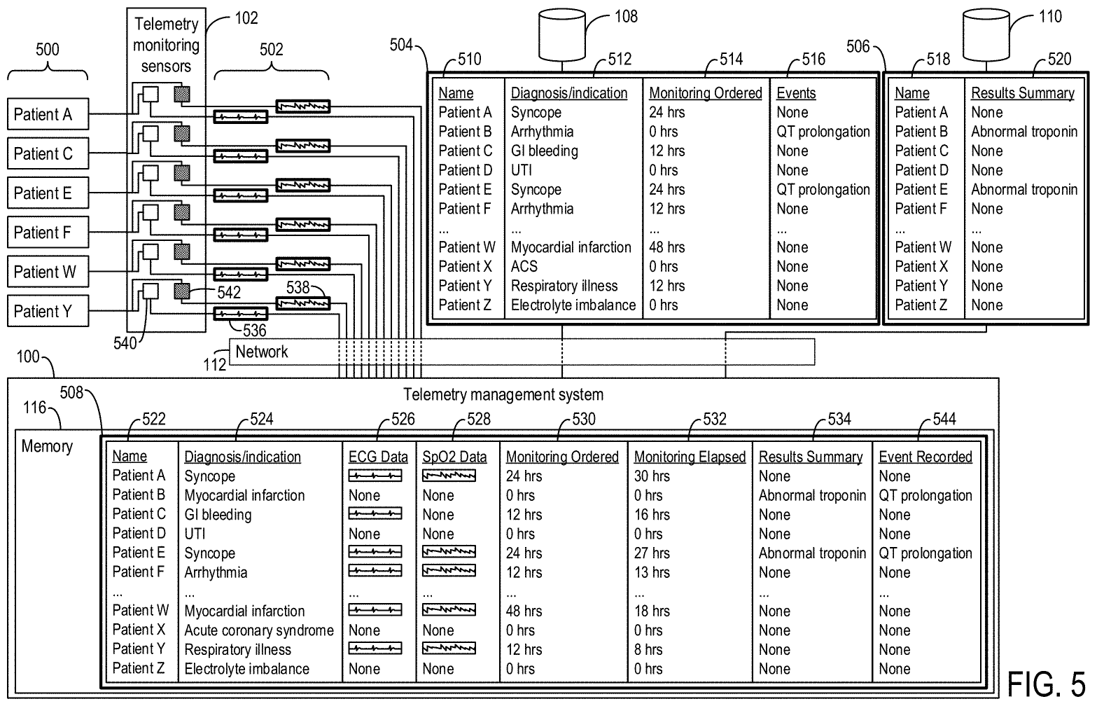

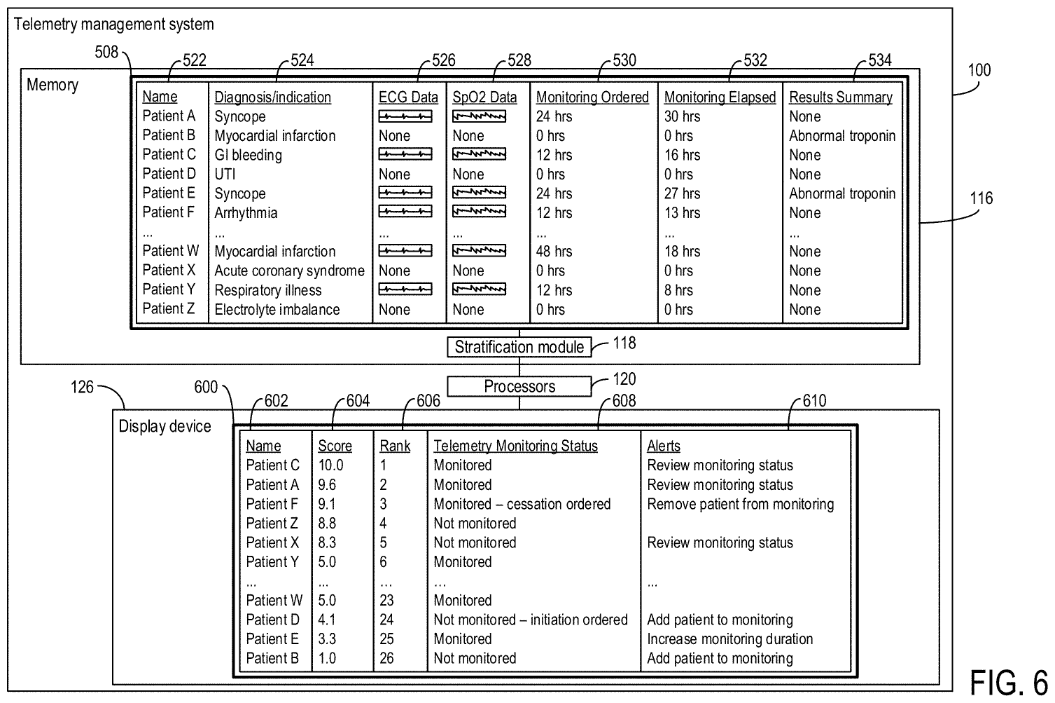

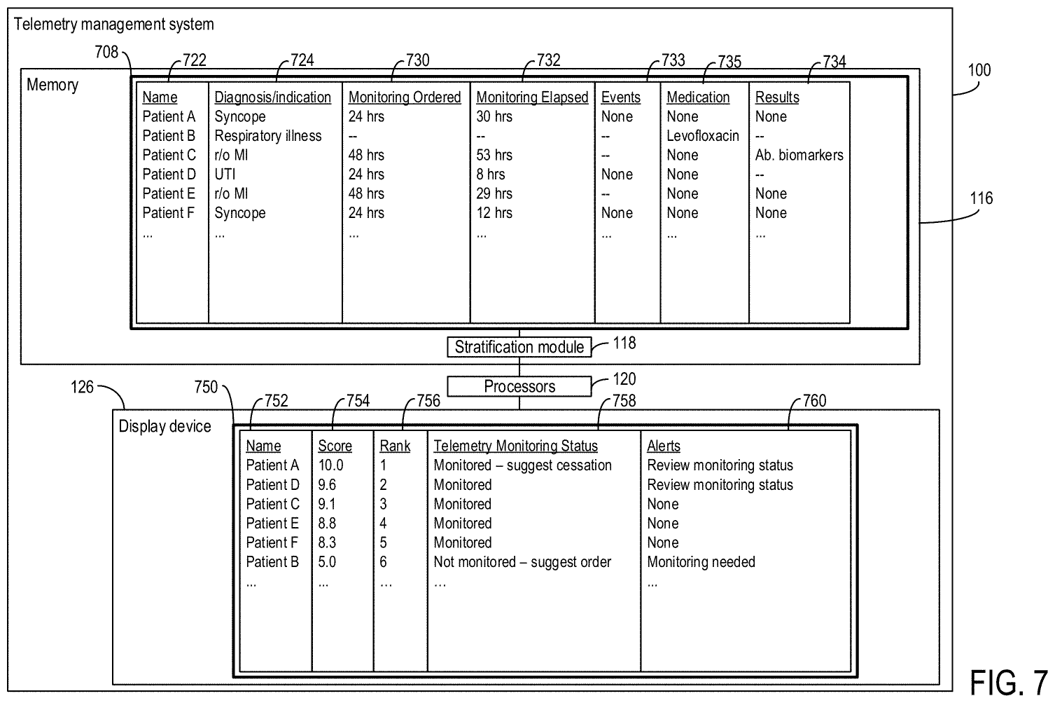

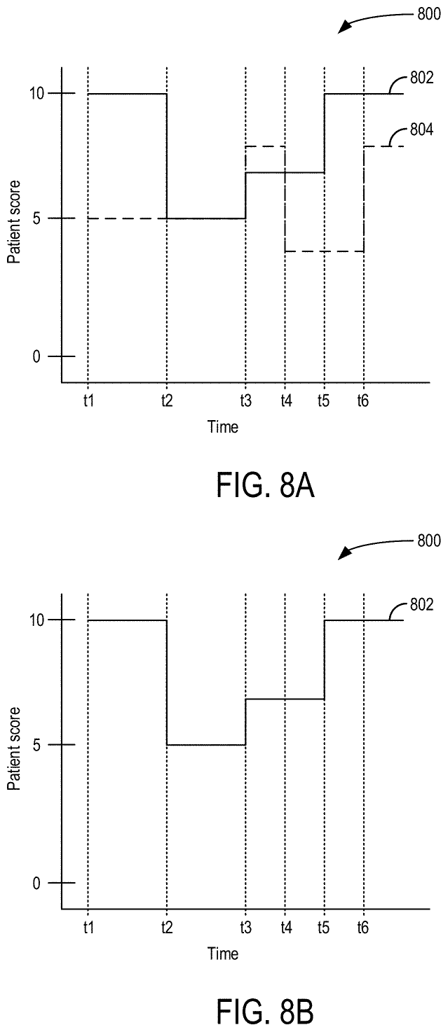

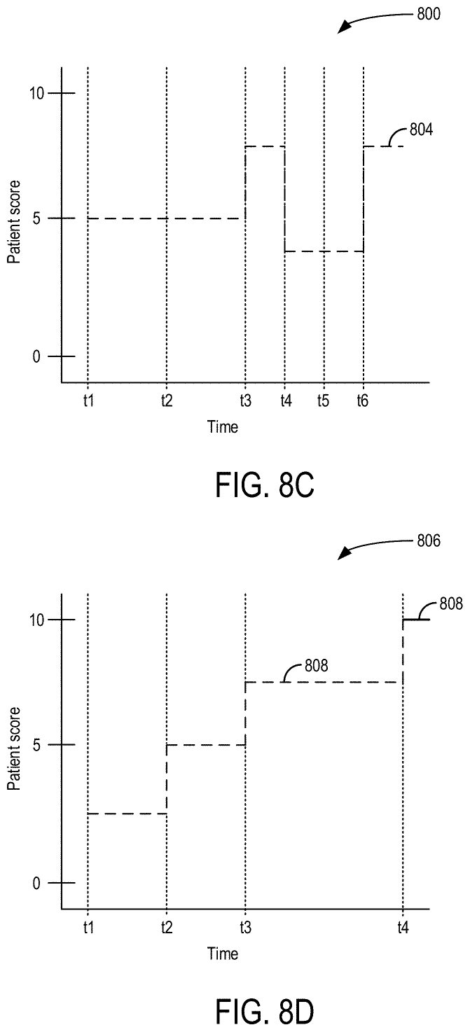

An example telemetry management system is shown in FIGS. 1A and 1B. The telemetry management system may be included in or associated with a medical facility and may be electronically coupled to one or more databases via a network to receive patient information for each admitted patient of the medical facility, as illustrated schematically by FIG. 5. The telemetry management system may generate a stratified patient list based on the acquired patient information and display the stratified patient list to care providers via a display device, as illustrated by the flowchart of FIG. 2 and as shown schematically by FIGS. 6 and 7. The stratified list ranks patients according to patient score, with patient score being determined at least in part by a telemetry monitoring status of each patient. The telemetry management system may determine the telemetry monitoring status of each patient based on whether a telemetry monitoring initial request or telemetry monitoring cessation request has been ordered and whether telemetry monitoring data is transmitted to the telemetry management system, as illustrated by the flowchart of FIG. 3. The telemetry management system then determines a score for each patient based on the patient information and patient telemetry monitoring status, as illustrated by FIGS. 4A-4B. Example patient scores determined by the telemetry management system for example patients are shown in FIGS. 8A-8D. Further, the telemetry management system may determine the telemetry monitoring score for patients that are not currently being monitored via telemetry monitoring, and have not received a telemetry monitoring order. For example, the telemetry management system may recommend, based on the telemetry monitoring score, whether or not patients should be placed on telemetry monitoring. The telemetry monitoring score may be based on patient information, such as diagnosis, medication orders, lab tests, and so forth.

By configuring the telemetry management system to score the patients based on the acquired patient information from the one or more databases with respect to monitoring guidelines/standards and generate the stratified patient list based on the patient scores, the telemetry management system provides an intuitive way for care providers to quickly classify patients and adjust patient telemetry monitoring priority. The telemetry management system may decrease the ranking of patients that may benefit from telemetry monitoring (e.g., move the patients to undergo telemetry monitoring toward the end of the stratified list) and may maintain or increase the ranking of patients that may receive less benefit from telemetry monitoring (e.g., move the patients to be removed from telemetry monitoring toward the beginning of the stratified list). Further, the telemetry management system may provide alerts for patients to indicate recommendations for initiation, cessation, or prolongation of telemetry monitoring based on the acquired patient information. The telemetry management system may reduce an amount of time and/or effort expended by care providers to determine a telemetry monitoring protocol for a large number of patients by automatically acquiring patient information from the one or more databases. As a result, efficiency of care may be increased, and telemetry resources may be reallocated to patients according to patient need.

FIG. 1A shows an example of a system for telemetry monitoring 10 including a telemetry management system 100 and telemetry monitoring sensors 102a and 102b.

As shown in FIG. 1A, a first patient 20a may be assigned to a first telemetry monitoring sensor 102a. The first telemetry monitoring sensor 102a may connected to a noninvasive blood pressure monitor (NIBM) 105 which monitors a blood pressure of the patient 20a and a respiratory rate (RR) monitor 103 which monitors a respiratory rate of the patient 20a. The telemetry monitoring sensor 102a may transmit information obtained from the sensors 103 and 105 to the telemetry management system 100 over a network 112. The information may be transmitted wirelessly so that the patient 20a is able to ambulate or move around using other means.

The second telemetry monitoring sensor 102b may connected to a peripheral capillary oxygen saturation (SpO2) sensor 106 which monitors a blood oxygen saturation of the patient 20b and an electrocardiogram (ECG) sensors 104 which monitors electrical activity of the patient 20b. The telemetry monitoring sensor 102b may transmit information obtained from the sensors 104 and 106 to the telemetry management system 100 over a network 112. The information may be transmitted wirelessly so that the patient 20b is able to ambulate or move around using other means.

FIG. 1B schematically shows an example telemetry management system 100 that may be implemented in a medical facility such as a hospital. Telemetry management system 100 includes resources (e.g., memory 116 and processor(s) 120) that may be allocated to store and execute a stratification algorithm stored in non-transitory memory of the telemetry management system 100. For example, as shown in FIG. 1B, stratification module 118 is stored within a non-transitory memory of memory 116 of the telemetry management system 100 and includes one or more stratification algorithms for generating a stratified patient list based on information acquired by the telemetry management system 100.

Memory 116 includes non-transitory data storage structures, which may include optical memory devices, magnetic memory devices, or solid-state memory devices, for storing programs and routines (e.g., stratification algorithms) executed by processors 120 to carry out various functionalities disclosed herein. Memory 116 may include any desired type of volatile and/or non-volatile memory such as, for example, static random access memory (SRAM), dynamic random access memory (DRAM), flash memory, read-only memory (ROM), etc. Processors 120 may be any suitable processor, processing unit, or microprocessor, for example. In the embodiment shown, processors 120 include central processing unit (CPU) 122 and graphics processing unit (GPU) 124. However, in some embodiments, processors 120 may include only CPU 122 or may include a combination CPU/GPU. In some embodiments, processors 120 may be a multi-processor system, and, thus, may include one or more additional processors (e.g., additional GPUs) that are identical or similar to each other and that are communicatively coupled via an interconnection bus.

Processors 120 are electronically coupled to display device 126 of the telemetry management system 100. Display device 126 may be a screen, monitor, mobile/smart device, or other suitable device configured to display an output of the telemetry management system 100 to a user (e.g., care provider). For example, display device 126 may display a user interface 128 according to an output of processors 120 (e.g., an output of CPU 122, GPU 124, or a combination thereof). User interface 128 may be a text-based interface in some embodiments. In other embodiments, user interface 128 may be a graphical user interface including virtual representations of buttons, icons, and the like, as well as contextual patient information. The telemetry management system 100 may further include one or more interface devices (e.g., mouse, keyboard, etc.) that may be utilized by a user of the telemetry management system 100 to interact with the user interface 128 of the telemetry management system 100 displayed at display device 126. In some embodiments, display device 126 may be a touchscreen configured to respond to a touch of the user in order to enable the user to interact with user interface 128.

As described above, processors 120 may execute stratification instructions (e.g., a stratification algorithm) stored by stratification module 118 and may display an output of the stratification algorithm at the display device 126. For example, the output of the stratification algorithm may be included within user interface 128. The processors 120 generate a stratified patient list via the instructions stored by the stratification module 118 based on patient information acquired by the telemetry management system 100 from one or more databases, as described further below.

The telemetry management system 100 may communicate electronically with one or more networked computing systems 130 within and/or external to the facility via network 112. Network 112 may be configured as a wired local area network (LAN), wireless LAN, wide area network (WAN), etc. The networked computing systems 130 may include respective display devices in order to view the stratified patient list generated by the telemetry management system 100. Each of the networked computing systems 130 may include a processor, memory, user input device, display (e.g., screen or monitor), and/or other subsystems and may be in the form of a desktop computing device, a laptop computing device, a tablet, a smart phone, or other device. Each of the networked computing systems 130 may be adapted to send and receive encrypted data and display information transmitted by the telemetry management system 100, including acquired patient information and the stratified patient list. The networked computing systems 130 may be located locally at the medical facility (such as in a nurses station or in the room of a patient) and/or remotely from the medical facility (such as a care provider's mobile device).

The telemetry management system 100 may be communicatively to one or more systems and databases for acquiring patient information (e.g., a list of patient names, diagnoses, etc.). The systems and databases may store and/or control a variety of hospital-, care provider-, and patient-related information, including but not limited to patient admission information (including date of admission and location of the patient within the medical facility), patient care protocols and workflows, and care provider information including which care providers are monitoring/treating which patients. As shown in FIG. 1B, the systems and databases may include (but are not limited to) a cardiology management system 150, a computerized physician order entry (CPOE) system 107, an electronic medical record (EMR) database 108, a laboratory information system 110, a pharmacy information system 109, and a protocols/standards system 111. Additional details about the systems and databases will be provided below.

Further, the telemetry management system 100 may be communicatively coupled to a plurality of telemetry monitoring sensors 102 and may be configured to receive electronic signals from the telemetry monitoring sensors 102. The telemetry monitoring sensors 102 may include ECG sensors 104, SpO2 sensors 106, RR sensors 103, non-invasive blood pressure (NIBP) sensors 105, and temperature sensors 101 configured to monitor respective patients undergoing telemetry monitoring. The telemetry monitoring sensors 102 may send output directly to the telemetry management system 100 and/or may send output to the systems and databases (e.g., EMR 108). For example, a plurality of telemetry monitoring sensors monitoring a first patient may be configured to send output to the telemetry management system 100, and the output acquired by the telemetry management system 100 may be processed by the stratification module 118 in order to determine a score of the first patient when generating the stratified patient list. In some embodiments, telemetry management system 100 may additionally receive diagnostic imaging information from one or more imaging modalities, such as ultrasound, CAT, MM, X-ray, etc., via a suitable system/database, such as via a radiology information system. Additionally, in some embodiments, telemetry management system 100 may receive patient lab results from laboratory information system (LIS) 110 (described further below) and/or medication information from the pharmacy information system 109.

The telemetry management system 100 may determine a score for each patient via the stratification module 118 based on the information acquired by the telemetry management system 100 from the systems and databases (e.g., EMR 108, LIS 110, cardiology management system 150), and telemetry monitoring sensors 102. For example, when a patient is admitted to the facility, the telemetry management system 100 may acquire patient diagnosis information from the systems and databases in order to determine the patient score.

In some embodiments, the score of newly admitted patients may be a same pre-determined score assigned to each newly admitted patient (e.g., a score of 5.0, in one non-limiting example). In other embodiments, the score of a newly admitted patient may be based on a severity of the diagnosis of the patient. For example, a patient admitted for myocardial infarction may receive a lower score (and may be located at a lower position within the stratified patient list) and thus a higher rank for the need for telemetry than a patient admitted for a urinary tract infection (UTI) and placed on telemetry monitoring. The telemetry management system 100 may obtain protocols/standards (e.g., from protocols/standards system 111) that may be accessed by stratification module 118 in order to determine the score of newly admitted patients based on patient diagnoses. For example, myocardial infarction may be associated with a first, lower score within the criteria, and UTI may be associated with a second, higher score within the criteria. In yet other embodiments, the score assigned to each newly admitted patient may be an average score (e.g., mean or median score) of all patients within the stratified patient list.

In some embodiments, the systems and databases may be external databases accessible by the telemetry management system 100 via a secured hospital interface (e.g., network 112). In other embodiments, the systems and databases may be local databases (e.g., housed on the telemetry management system 100). The systems and databases may include patient information (e.g., patient names, diagnoses, etc.) stored in mass storage device(s) configured to communicate with secure channels (e.g., HTTPS and TLS), and store data in encrypted form. The EMR database 108 may be configured to control access to patient electronic medical records such that only authorized healthcare providers may edit the electronic medical records. An EMR for a patient may include patient demographic information, family medical history, past medical history, lifestyle information, preexisting medical conditions, current medications, current lab test results, allergies, surgical history, past medical screenings and procedures, past hospitalizations and visits, etc.

Cardiology management system (CMS) 150 may collect, store, analyze, and/or manage information from medical devices (e.g., resting ECG, stress or exercise ECG, Holter ECG, patient monitoring, etc. medical devices) used to detect and diagnose cardiac-related issues of patients. In some examples, cardiology management system 150 may interface with EMR 108 in order to update patient electronic medical records to indicate the detected and/or diagnosed cardiac-related issues. In some examples, while in the ED, a suspected myocardial infarction patient could have a resting ECG performed on them. If the device and CMS detects an abnormality, this could be an indication to the physician that telemetry should be considered to be administered or is part of the guideline or protocol to be followed. The telemetry management system may periodically query the CMS 150 for updates/triggering events to be accounted for and included in the indexing scheme.

CPOE system 107 may receive orders from care providers and communicate the orders to other care providers or department devices responsible for fulfilling the orders. The orders may include instructions for the treatment of patients, such as procedures to be performed, medications to be administered, operational sequences to be followed, and the like.

Laboratory information system (LIS) 110 may include one or more computing devices associated with an on-site or off-site laboratory that performs lab tests on patient specimens sent to the lab by the care provider(s). The one or more computing devices may include resources (e.g., memory and processors) allocated to manage various aspects of the laboratory procedures, such as managing/assisting with tagging of incoming specimens (e.g., with patient and care provider information, test(s) to be conducted on the specimen, and so forth), tracking specimens (e.g., in storage, being processed), generating reports of test results, and the like. Accordingly, the LIS may interface directly with various laboratory equipment, such as mass spectrometers, chromatographers, analyzers, etc., and thus may have knowledge of which specimens are currently being tested, the results of such tests, and the performance status of the various pieces of equipment. The LIS may also provide additional data which is not communicated to the EMR which may help determine or predict patient status.

Pharmacy information system 109 may include one or more computing devices associated with an on-site or off-site pharmacy that fills prescriptions as ordered by care providers. The one or more computing devices may include resources (e.g., memory and processors) allocated to receive prescription requests and communicate the requests with pharmacy staff, track prescription fill status, notify an ordering care provider when a prescription is available, and so forth.

In some examples, the telemetry management system 100 may interface with one or more other databases, such as database 151, for on-going data processing, data storage, analytics, reporting, etc. In the embodiment shown, database 151 is a database located within the patient facility. However, in other embodiments, the database 151 may utilize a cloud-based data architecture and may not be located within the patient facility.

Telemetry management system 100 may periodically query the systems and databases described herein (e.g., cardiology management system 150, CPOE 107, EMR 108, LIS 110, pharmacy information system 109, and protocols/standards system 111) to acquire updated information (e.g., patient information, protocols, etc.) for managing (e.g., forming, updating, etc.) the stratified patient list. As one example, telemetry management system 100 may periodically query LIS 110 to acquire updated lab test results for patients within the stratified patient list and/or LIS 110 may push lab test results to telemetry management system 100. Once the test results are available, the telemetry management system 100 may utilize the test results in order to update the patient score of the corresponding patients within the stratified list. Likewise, telemetry management system 100 may periodically query pharmacy information system 109 to acquire medication information for patients within the stratified patient list and/or pharmacy information system 109 may push mediation orders/prescription fills to telemetry management system 100. If a new medication is prescribed/filled for a patient, the telemetry management system 100 may utilize the prescription information in order to update the patient score of the corresponding patients within the stratified list. The telemetry management system 100 may then adjust the rank of the patients within the stratified patient list based on the updated patient scores. For example, a patient may be admitted to the facility with a diagnosis having a relatively low likelihood of increasing in severity (e.g., a UTI). However, after an amount of time has elapsed, lab results transmitted to the telemetry management system 100 may indicate a change in the condition of the patient (e.g., where the lab results indicate one or more tested biomarker levels are outside a normal or healthy range). For example, lab results may indicate abnormal cardiac biomarkers such as troponin levels which may be indicative of an acute myocardial infarction. As a result, the telemetry management system 100 may reduce the score of the patient and may set an alert for a care provider to adjust a telemetry monitoring status of the patient accordingly (e.g., initiate telemetry monitoring of the patient or increase a duration of telemetry monitoring of the patient).

The telemetry management system 100 may integrate data from the telemetry monitoring sensors 102 (e.g., data generated by the telemetry monitoring sensors 102) and the systems and databases via the stratification module 118 to enable smart, automated management of telemetry monitoring of the patients within the facility. The stratification module stratifies patients by score, with the score based on the patient information acquired from the systems and databases in combination with output received by the telemetry monitoring sensors 102. Patients having a higher score (e.g., patients near a top or beginning of the stratified patient list) may be flagged with an alert in the stratified patient list indicating that cessation of telemetry monitoring is recommended.

In this way, low risk patients (e.g., patients less likely to benefit from telemetry monitoring) may be quickly identified, and a care provider may adjust patient treatment accordingly (e.g., remove the patients from telemetry monitoring). Additionally, deteriorating patients (as indicated by updated patient information acquired from the systems and databases) may trigger via the stratification module a reduction in patient score that places the deteriorating patients lower in the stratified patient list (e.g., toward a bottom or end of the stratified patient list) in order to quickly indicate that telemetry monitoring of the deteriorating patients may be more beneficial relative to the lower risk patients. The stratified patient list generated by the stratification module 118 of the telemetry management system 100 may quickly provide recommendations for cessation or initiation of telemetry monitoring (e.g., identifying patients that may need telemetry monitoring but are not currently being monitored) for a large number of patients (e.g., 400 patients) and may decrease an effort expended by care providers, increasing an efficiency of care. Further, providing the stratified patient list may enable care providers to quickly identify low risk patients that may be removed from telemetry monitoring, freeing up facility resources and increasing productivity while reducing care provider alarm fatigue.

In some examples, each patient score/rank may be determined or interpreted in light of one or more treatment protocols or guidelines stored on and obtained from protocols/standards system 111. Protocols/standards system 111 may store telemetry monitoring protocols or standards for a plurality of different patient conditions or indications. The telemetry monitoring protocols or standards may indicate, for each of a plurality of indications, how long a patient presenting with that indication is to be monitored, what potential diagnoses or treatments may be associated with different monitoring outcomes, and so forth. For example, if a patient is admitted to a medical facility with an indication of syncope, the telemetry management system 100 may retrieve telemetry monitoring guidelines for patients presenting with syncope, which may dictate how long the patient is to be monitored with which telemetry monitoring sensors. The telemetry management system may then compare the amount of time the patient has been monitored to the recommended amount of monitoring time and update the patient score based on the elapsed versus recommended time.

In some embodiments, telemetry management system 100 may utilize machine learning models to help predict patient risk and determine patient score (e.g., patient telemetry cessation score). Based on predicted outcomes, telemetry management system 100 may generate patient scores to adjust patient ranking within the stratified patient list according to the machine learning models.

As an example, a testing model may use current patient parameters (e.g., current diagnosed conditions, vital signs, length of stay at the medical facility, demographic information, medications, recent telemetry events (e.g., abnormal heart rhythms or high heart rate), etc., acquired by the telemetry management system 100 from the systems and databases and telemetry monitoring sensors 102) as inputs, and based on the inputs, the testing model may output patient score (e.g., patient telemetry cessation score), which may be communicated to the care providers via the stratified patient list.

The testing model may be a machine learning model or other deep learning model that is trained using information from past patients and known patient outcomes for those past patients. For example, the testing model may be trained with a plurality of training datasets, where each training dataset is specific to a respective patient and includes patient parameters for that patient and known outcomes for that patient. The patient parameters may include demographic information (e.g., age, sex, nationality, etc.) for that patient, current and/or past diagnosed conditions for that patient, patient lifestyle information (e.g., travel history, drug or alcohol use, exercise habits), current vital/health signs (e.g., heart rhythm, blood pressure, heart rate, respiration rate, sepsis, alertness, previous ECG, etc.) for that patient, and so forth. The known patient outcomes may include, for each respective patient, a telemetry monitoring duration of that patient. In some examples, the known patient outcomes (e.g., an amount of time in which the patients were monitored via telemetry monitoring) may be classified by whether the telemetry monitoring cessation of the patient occurred prematurely (e.g., prior to a resolution of a patient condition for which telemetry monitoring was ordered).

For example, the patient may be admitted to the facility with a diagnosis of syncope. The telemetry management system 100 may acquire the diagnosis from the CPOE system 107, cardiology management system 150, and/or EMR database 108 and may input relevant patient parameters into the testing model (e.g., patient vital signs, patient demographic information, current diagnosis, recent telemetry events, medications, etc.) and the testing model may output a score for the patient based on population wide information in order to rank the patient within the stratified patient list and determine alerts to display for the patient. In this case, if the patient has a history of heart degradation, the patient's medical history may have been noted in the patient's EMR. The testing model may then output an initial, lower score for the patient relative to patients that do not have a history of heart degradation. Further, the telemetry management system 100 may adjust the patient score and/or provide alerts based on known patient outcomes for past patients to provide a recommended telemetry monitoring duration for the newly admitted patient. For example, hospital guidelines (e.g., retrieved from protocols/standards system 111) may recommend 24 hours of telemetry monitoring for patients admitted with syncope. However, based on the known patient outcomes for past patients (e.g., patients having similar patient parameters such as age, medications, diagnosis, gender, diagnostic testing results, etc. relative to the newly admitted patient), the telemetry management system 100 may provide an alert recommending a telemetry monitoring duration that is longer or shorter than 24 hours for the newly admitted patient. In the case that a longer telemetry monitoring duration is recommended, the telemetry management system 100 may reduce the score of the patient (e.g., in order to place the patient at a lower ranking within the stratified patient list), and in the case that a shorter telemetry monitoring duration is recommended, the telemetry management system 100 may increase the score of the patient (e.g., in order to place the patient at a higher ranking within the stratified patient list). The telemetry management system 100 may thus provide recommendations for telemetry monitoring according to population patterns determined via the machine learning models that may otherwise be inaccessible or go unnoticed to care providers. In some embodiments, the machine learning models may be included within the stratification module 118.

In some embodiments, a separate alert notification system 132 may communicate with telemetry management system 100 via network 112 (or other suitable connection). The alert notification system 132 may include resources (e.g., memory and one or more processors) allocated to distributing alerts generated by telemetry management system 100. For example, while telemetry management system 100 may include a display device 126 for displaying the alerts described herein, care provider interaction with the display device 126 may be limited. Thus, to ensure the alerts generated by the telemetry management system 100 are distributed to all relevant care providers, the alert notification system 132 may push alerts to the appropriate devices of the networked computing systems 130. Further, the alert notification system 132 may include its own display device on which alerts may be displayed.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

One or more of the devices described herein may be implemented over a cloud or other computer network. For example, telemetry management system 100 is shown in FIG. 1B as constituting a single entity, but it is to be understood that telemetry management system 100 may be distributed across multiple devices, such as across multiple servers, with at least one of the servers including the stratification module 118.

While not specifically shown in FIG. 1B, additional devices described herein (CPOE system 107, EMR database 108, pharmacy information system 109, LIS 110, protocols/standards system 111, cardiology management system 150, and networked computing systems 130) may likewise include user input devices, display devices, memory, and processors similar to communication memory 116, processors 120, and display device 126 described above, and thus the description of memory 116, processors 120, and display device 126 likewise applies to the other devices described herein. As an example, the networked computing systems 130 may store user interface templates in memory that include placeholders for relevant information stored and output by telemetry management system 100. For example, one or more of networked computing systems 130 may store a user interface that displays the stratified patient list output by the telemetry management system 100. The user input devices of the networked computing systems 130 may include keyboards, mice, touch screens, microphones, or other suitable devices.

Turning now to FIG. 2, a flow chart of a method 200 for managing a telemetry protocol via a telemetry management system is shown, according to an exemplary embodiment. Method 200 may be implemented by the telemetry management system 100 shown in FIG. 1B. In some embodiments, method 200 may be implemented as executable instructions in a stratification module of a telemetry management system, such as the stratification module 118 of FIG. 1B.

At 202, a patient index is generated, with the patient index including a list of patients and patient information. In some embodiments, the patient index may be generated (e.g., compiled) by the telemetry management system using information acquired from various databases, as described below. In other embodiments, the patient index may be compiled by an external computing system (e.g., the one or more databases) and transmitted to the telemetry management system. The patient index may be stored in a memory of the telemetry management system (e.g., memory 116 shown by FIG. 1B and described above) and may be accessed by a stratification module of the telemetry management system (e.g., stratification module 118 shown by FIG. 1B and described above).

In some embodiments, as indicated at 204, the patient information acquired at 202 may include patient records acquired from one or more databases and/or systems. The one or more databases and/or systems may include an electronic medical records (EMR) database (e.g., EMR database 108), a computerized physician order entry (CPOE) system (e.g., CPOE system 107), a cardiology management system (e.g., cardiology management system 150), a pharmacy information system (e.g., pharmacy information system 109), a radiology information system (RIS), etc. The patient records may include parameters such as patient name, diagnosis, facility admission time, prior, current, and/or ordered medications, treatment plan, etc. The databases/systems may transmit the patient information including the patient records to the telemetry management system. For example, the telemetry management system may periodically query the databases in order to acquire updated patient information (e.g., updates to patient records) from the databases. In other embodiments, the databases may automatically transmit updated patient records to the telemetry management system as the patient records are modified. For example, a clinician may update a diagnosis of a patient in the patient records stored within the databases, and the databases may automatically transmit the updated diagnosis of the patient to the telemetry management system to update the diagnosis stored in the memory of the telemetry management system.

In some embodiments, as indicated at 206, the patient information acquired at 202 may include lab results acquired from a lab information system (LIS). The LIS may be the LIS 110 shown by FIG. 1B and described above, in some embodiments. The LIS may transmit data including the patient lab results to the telemetry management system. Patient lab results may include, for example, patient cardiac enzyme levels, patient electrolyte levels, complete blood count, etc. In some embodiments, the lab results acquired from the LIS may result from tests performed after admission of the patient to the facility. As one example, a patient may be admitted for a condition (e.g., syncope), and a clinician may order one or more lab tests to be performed while the patient is receiving treatment within the facility. The lab results acquired from the LIS may include results from the lab tests (e.g., in order to indicate a progression of treatment of the patient). In some embodiments, the lab results acquired from the LIS may include the results of lab tests performed prior to admission of the patient to the facility.

In some embodiments, as indicated at 208, the patient information acquired at 202 may include telemetry monitoring data acquired from telemetry monitoring sensors. In one embodiment, the telemetry monitoring sensors may be the telemetry monitoring sensors 102 shown by FIG. 1B and described above. The telemetry monitoring sensors may transmit data such as patient heart rate, patient peripheral capillary oxygen saturation, patient events (e.g., irregular heart rhythms), patient respiration rate, patient blood pressure, patient heart rhythm, patient temperature, etc., to the telemetry management system for each patient coupled to the telemetry monitoring sensors. As one example, each patient undergoing telemetry monitoring may be coupled to a separate set of telemetry monitoring sensors (e.g., an ECG sensor, SpO2 sensor, NIBP sensor, respiration sensor, temperature sensor, etc.), and the telemetry management system may acquire telemetry monitoring data from each set of telemetry monitoring sensors. The telemetry monitoring data acquired from each patient may be associated with the corresponding name of the patient within the patient index (e.g., within a data array of the patient index).

In some embodiments, as indicated at 209, the patient information acquired at 202 may include protocol and/or indication information to be followed during care of the patient at the medical facility. As explained above, the protocol information may include protocols or standards for telemetry monitoring for a plurality of different indications or patient conditions. Thus, the patient information may include current patient indication or condition (e.g., as diagnosed by a care giver and entered into the CPOE system and/or EMR) and the telemetry monitoring protocol(s) for that indication or condition. The telemetry monitoring protocols may include recommended monitoring duration and monitoring sensors, for example.

In some embodiments, the telemetry management system may generate the patient index by compiling the information transmitted to the telemetry management system from the systems/databases and/or telemetry monitoring sensors. As one example, the telemetry management system may receive patient information including a list of patient names and diagnoses/indications from the databases, patient information including lab results from the LIS and medications from the pharmacy information system, and patient information including telemetry monitoring data (or lack thereof) from the telemetry monitoring sensors. The telemetry management system may store the patient information from the systems/databases and telemetry monitoring sensors separately in a memory of the telemetry management system, and may combine the patient information from each of the sources in order to form the patient index. The patient index formed by the telemetry management system in this way may include the list of patient names, patient diagnoses, patient lab results, and telemetry monitoring data for each patient coupled to telemetry monitoring sensors. In some embodiments, the patient index may be a data array (e.g., data table) including data (e.g., the patient diagnoses, patient lab results, and telemetry monitoring data) for a plurality of different patients. For example, the patient index may be an array including a plurality of subarrays, where, for each patient, a different patient information parameter is stored in each subarray (e.g., patient name stored in a first subarray, patient diagnosis stored in a second subarray, patient lab results stored in a third subarray, telemetry monitoring data stored in a fourth subarray, etc.).

The patient index may thus be an array of patient information, with a first subarray including the list of patient names, a second subarray including the patient diagnoses, a third subarray including the lab results, and a fourth subarray including the telemetry monitoring data. Additional subarrays may include medications (current or ordered), limit events (which may include telemetry monitoring data reaching high or low limits, such as when heart rate exceeds an upper threshold), current duration of telemetry monitoring, and recommended or ordered monitoring duration.

At 210, telemetry monitoring status is determined for each patient in the patient index. Determining the telemetry monitoring status for each patient in the patient index may include determining whether telemetry monitoring data is transmitted from telemetry monitoring sensors to the telemetry management system for each patient. For example, during conditions in which the patient information acquired at 202 includes telemetry monitoring data for a patient, the telemetry management system may determine that the patient is undergoing telemetry monitoring. Further, determining the telemetry monitoring status for each patient in the patient index may include, for each patient, determining whether a telemetry monitoring cessation request or a telemetry monitoring initiation request has been made, as illustrated by the flowchart of method 300 of FIG. 3, described in further detail below.

At 212, a score is determined for each patient in the patient index based on patient information and telemetry monitoring status. Determining the score for each patient in the patient index based on the patient information and telemetry monitoring data may include, for each patient, determining whether a triggering event has occurred (e.g., whether an indication of degradation of patient condition has occurred), determining whether telemetry monitoring cessation and/or initiation has occurred and a duration of telemetry monitoring (if applicable), and determining whether patient condition parameters are within a pre-defined telemetry monitoring criteria. In some embodiments, for each patient admitted to the facility, the patient may be assigned an initial score based on the patient information (e.g., based on a diagnosis of the patient, an average score of all patients within the patient index, etc., as described above), and the initial score may be adjusted (e.g., updated) by the telemetry management system at 212 according to the methods described herein. The determination of the score for each patient is described in further detail below with reference to the flowchart illustrating method 400 shown by FIGS. 4A-4B. In some examples, patients that are not currently being monitored via the telemetry monitoring sensors described herein may also be given a score that is based on indication/diagnosis, lab results, medications, and protocols/standards. In this way, if the patient indication/diagnosis, lab results, and/or medications for a given patient indicate that telemetry monitoring should be initiated, the score assigned to that patient may reflect the need for telemetry monitoring for that patient, and appropriate care givers may be notified (as will be explained in more detail below).

At 214, a stratified patient list is formed based on the score of each patient, and the stratified patient list is displayed at a display device. In some embodiments, the display device may be the display device 126 of the telemetry management system 100 shown by FIG. 1B and described above and/or other suitable display devices, such as display devices associated with care provider devices and/or an alert notification system. The stratified patient list may include the patient names ranked by score. In some embodiments, patients having higher scores may be displayed at a higher position in the stratified patient list (e.g., toward a beginning or top of the stratified patient list). In other embodiments, patients having higher scores may be displayed at a lower position in the stratified patient list. However, in each embodiment, the patients within the stratified patient list are ranked sequentially by score either in ascending order (e.g., with patients having higher scores positioned higher) or descending order (e.g., with patients having lower scores positioned lower). In some embodiments, a care provider may interact with a user interface of the telemetry management system (e.g., user interface 128 of FIG. 1B) in order to adjust the display of the stratified patient list from the ascending order to the descending order, or vice versa.

In some embodiments, as indicated at 216, forming the stratified patient list based on the score of each patient and displaying the stratified patient list at the display device at 214 may include displaying alerts assigned to the patients within the stratified patient list at the display device. For example, as described further below with reference to the method 400 illustrated by the flow chart of FIGS. 4A-4B, patients within the patient index may be assigned alerts (e.g., alerts may be set for one or more patients) for care provider review based on various treatment parameters and patient condition while the score is determined for each patient at 212. In some embodiments, patient alerts may be displayed alongside the patient names within the stratified patient list (e.g., as a separate field alongside the stratified patient list). For example, a first patient within the stratified patient list may be assigned an alert indicating that a review of the telemetry monitoring status of the first patient is recommended. The alert assigned to the first patient may be displayed alongside the name of the first patient at the location of the first patient within the stratified patient list (e.g., as illustrated by FIG. 6 and described below). Alerts may include indicators to add a patient to telemetry monitoring (e.g., recommend ordering initiation of patient telemetry monitoring), remove a patient from telemetry monitoring (e.g., recommend ordering cessation of patient telemetry monitoring), review the telemetry monitoring status of a patient (e.g., recommend a closer evaluation of the condition of the patient to determine whether telemetry monitoring of the patient is desired), and/or adjust a duration of telemetry monitoring (e.g., increase or decrease a duration of patient telemetry monitoring). Conditions that may result in the assignment of an alert to a patient within the stratified patient list are described below with reference to FIGS. 4A-4B.

In some embodiments, as indicated at 218, forming the stratified patient list based on the score of each patient and displaying the stratified patient list at the display device at 214 may include displaying patient telemetry monitoring status for patients within the stratified patient list at the display device. As one example, displaying the patient telemetry monitoring status may include displaying whether each patient is either currently undergoing telemetry monitoring or not currently undergoing telemetry monitoring. Displaying the patient telemetry monitoring status may additionally include displaying whether a telemetry monitoring cessation order or telemetry monitoring initiation order has been made for each patient within the stratified patient list. In some embodiments, the patient telemetry monitoring status for each patient may be displayed adjacent to the name of the patient in a table or other display format, as illustrated by the embodiment shown by FIG. 6 and described further below.

In some examples, method 200 optionally includes sending patient monitoring data, including the patient scores, stratified patient list, alerts, and patient telemetry monitoring statuses, to one or more appropriate devices for reporting and analytics. For example, as explained previously, patient score may be determined using artificial intelligence based algorithms, such as machine learning or other deep learning algorithms. In such examples, the patient monitoring data may be used to update and refine the algorithms. Further, the patient monitoring data may be compiled in order to facilitate determination of monitoring compliance, efficacy, variability, and other analytics that may drive adjustments to telemetry monitoring protocols. In some examples, the reporting and/or analytics may be performed locally, e.g., by the telemetry management system, and the results of the reporting and/or analytics may be saved locally, displayed locally, and/or sent to other devices for storage, display, etc.

Referring now to FIG. 3, a flow chart of a method 300 for determining a patient telemetry monitoring status via a telemetry management system is shown, according to an exemplary embodiment. In some embodiments, method 300 may be executed by the telemetry management system (e.g., telemetry management system 100 of FIG. 1B) for each patient within the patient index, as indicated at 210 of method 200 of FIG. 2, described above. Although method 300 may be described herein as applied to a single patient within the patient index, it should be understood that the telemetry management system may execute method 300 for each patient within the patient index. Method 300 may be implemented as executable instructions in a stratification module of the telemetry management system, such as the stratification module 118 of FIG. 1B.

At 302, a determination is made of whether a telemetry monitoring cessation order has been received. The determination of whether a telemetry monitoring cessation order is received may include determining whether a telemetry monitoring cessation order has been placed for a patient based on patient information acquired by the telemetry management system (e.g., the patient information acquired by the telemetry management system at 202 of method 200 of FIG. 2). For example, during conditions in which a care provider determines that telemetry monitoring of a patient is no longer desired, the care provider may update the CPOE and/or electronic medical record of the patient (e.g., the EMR stored in an EMR database electronically coupled to the telemetry management system, which may be the EMR database 108 shown by FIG. 1B and described above) to include a telemetry monitoring cessation order. The telemetry management system may query the EMR and/or CPOE of the patient in order to determine whether a telemetry monitoring cessation order has been placed, or the telemetry management system may receive a notification from the EMR and/or CPOE system that a telemetry monitoring cessation order has been placed. In other examples, when a care provider determines that telemetry monitoring of a patient is no longer needed (e.g., the patient has been monitored for the recommend amount of time), the care provider may enter an input to a computing device (e.g., via a user interface of the telemetry management system) and the telemetry management system may be notified of the cessation order directly (e.g., without receiving the cessation order via the EMR).

If a telemetry monitoring cessation order is received at 302, a determination is made at 304 of whether telemetry monitoring data is currently received. Telemetry monitoring data may include signals (e.g., electronic signals) transmitted to the telemetry management system by one or more telemetry monitoring sensors coupled to a patient (e.g., an ECG sensor and/or SpO2 sensor, which may be the ECG sensors 104 and SpO2 sensors 106 shown by FIG. 1B and described above). In some embodiments, the telemetry monitoring data may be received by the telemetry management system via a network (e.g., network 112 shown by FIG. 1B and described above). As described above with reference to method 200 shown by FIG. 2, the telemetry management system may store the telemetry monitoring data for patients within a patient index. In making the determination at 302 for a single patient within the patient index, the telemetry management system may reference the patient index (e.g., read or scan the applicable portion of the patient index, such as a sub-array designated to store telemetry monitoring data associated with the patient) in order to determine whether telemetry monitoring data is currently received for the patient (e.g., whether telemetry monitoring data is being received at the time that the determination is made at 304).

If telemetry monitoring data is not currently received at 304, patient telemetry monitoring status is updated at 306 to indicate that telemetry monitoring cessation is completed. The indication that telemetry monitoring cessation is completed may be stored in a memory of the telemetry management system. In some embodiments, the patient telemetry monitoring status may be stored within a sub-array of the patient index associated with the patient (e.g., linked to the sub-array including the patient name), and at 306 the telemetry management system may update the sub-array including the patient telemetry monitoring status to indicate that telemetry monitoring cessation is completed. In some embodiments, the patient telemetry monitoring status may additionally include an indication that the patient is not undergoing telemetry monitoring (e.g., due to the absence of telemetry monitoring data being received by the telemetry management system).

However, if telemetry monitoring data is currently received at 304, patient telemetry monitoring status is updated at 308 to indicate that telemetry monitoring cessation is incomplete. The indication that telemetry monitoring cessation is incomplete may be stored in the memory of the telemetry management system (e.g., stored within the sub-array of the patient index associated with the patient). At 308 the telemetry management system may update the sub-array including the patient telemetry monitoring status to indicate that telemetry monitoring cessation is incomplete. In some embodiments, the patient telemetry monitoring status may additionally include an indication that the patient is undergoing telemetry monitoring (e.g., due to the telemetry monitoring data being received by the telemetry management system).

In one example, prior to execution of method 300, a care provider may have ordered a telemetry monitoring cessation request for the patient. However, at the time the determination is made at 304, the cessation of the telemetry monitoring of the patient may not yet have been performed (e.g., performed by care provider such as a nurse, clinician, etc.) and as a result, telemetry monitoring data from sensors coupled to the patient may still be received by the telemetry management system. As a result, the telemetry management system may update the patient telemetry monitoring status in the patient index at 308 to indicate that the telemetry monitoring cessation is incomplete (e.g., the patient is still undergoing telemetry monitoring, despite the telemetry monitoring cessation request/order). By updating the telemetry monitoring status of the patient, the telemetry management system may utilize the telemetry monitoring status in order to provide graphical reminders, alerts, or other indicators to prompt the care provider to perform the cessation of telemetry monitoring, as illustrated by the embodiment shown below with reference to FIG. 6.

Returning now to 302, if a telemetry monitoring cessation order is not received, a determination is made at 310 of whether a telemetry monitoring initiation order is received. The determination of whether a telemetry monitoring initiation order is received may include determining whether a telemetry monitoring initiation order has been placed for a patient based on patient information acquired by the telemetry management system. For example, during conditions in which a care provider determines that initiation of telemetry monitoring of a patient is desired, the care provider may update the electronic medical record of the patient to include a telemetry monitoring initiation order. The telemetry management system may query the EMR and/or CPOE of the patient in order to determine whether the EMR or CPOE includes the telemetry monitoring initiation order. In other examples, if a telemetry monitoring imitation order is placed by a care provider, the EMR and/or CPOE system may push a notification of the order to the telemetry management system. In other examples, when a care provider determines that telemetry monitoring of a patient is desired, the care provider may enter an input to a computing device (e.g., via a user interface of the telemetry management system) and the telemetry management system may be notified of the initiation order directly (e.g., without receiving the initiation order via the EMR).

If a telemetry monitoring initiation order is received at 310, a determination is made at 312 of whether telemetry monitoring data is currently received. The determination of whether telemetry monitoring data is currently received at 312 may be similar to the determination described above at 304.

If telemetry monitoring data is not currently received at 312, patient telemetry monitoring status is updated at 314 to indicate that telemetry monitoring initiation is incomplete. The indication that telemetry monitoring initiation is incomplete may be stored in the memory of the telemetry management system (e.g., the patient telemetry monitoring status may be stored within the sub-array of the patient index associated with the patient, as described above). The telemetry management system may update the sub-array including the patient telemetry monitoring status to indicate that telemetry monitoring initiation is incomplete. In some embodiments, the patient telemetry monitoring status may additionally include an indication that the patient is not undergoing telemetry monitoring (e.g., due to the absence of telemetry monitoring data being received by the telemetry management system).

In one example, prior to execution of method 300, a care provider may have ordered a telemetry monitoring initiation request for the patient. However, at the time the determination is made at 312, the initiation of the telemetry monitoring of the patient may not yet have been performed (e.g., performed by care provider such as a nurse, clinician, etc.) and as a result, telemetry monitoring sensors (e.g., sensors 102 shown by FIG. 1B and described above) may not yet be coupled to the patient (or, the sensors may not yet be configured to output electronic signals to the telemetry management system). As a result, the telemetry management system may update the patient telemetry monitoring status in the patient index at 314 to indicate that the telemetry monitoring initiation is incomplete (e.g., the patient is not yet undergoing telemetry monitoring, despite the telemetry monitoring initiation request/order). By updating the telemetry monitoring status of the patient, the telemetry management system may utilize the telemetry monitoring status in order to provide graphical reminders, alerts, or other indicators to prompt the care provider to perform the initiation of telemetry monitoring, similar to the embodiment shown below with reference to FIG. 6.

However, if telemetry monitoring data is currently received at 312, patient telemetry monitoring status is updated at 316 to indicate that telemetry monitoring initiation is complete. The indication that telemetry monitoring initiation is complete may be stored in the memory of the telemetry management system (e.g., stored within the sub-array of the patient index associated with the patient, as described above). At 316 the telemetry management system may update the sub-array including the patient telemetry monitoring status to indicate that telemetry monitoring initiation is complete. In some embodiments, the patient telemetry monitoring status may additionally include an indication that the patient is undergoing telemetry monitoring (e.g., due to the telemetry monitoring data being received by the telemetry management system). However, if telemetry monitoring data is currently received at 312, patient telemetry monitoring status is updated at 316 to indicate that telemetry monitoring initiation is complete. The indication that telemetry monitoring initiation is complete may be stored in the memory of the telemetry management system (e.g., stored within the sub-array of the patient index associated with the patient). At 316 the telemetry management system may update the sub-array including the patient telemetry monitoring status to indicate that telemetry monitoring initiation is complete. In some embodiments, the patient telemetry monitoring status may additionally include an indication that the patient is undergoing telemetry monitoring (e.g., due to the telemetry monitoring data being received by the telemetry management system).

Returning to 310, if a telemetry monitoring initiation order is not received, the patient telemetry monitoring status is maintained at 318. Maintaining the telemetry monitoring status may include not updating/modifying the telemetry monitoring status in the patient index. In some embodiments, the telemetry monitoring status of a patient may indicate that the patient is currently undergoing telemetry monitoring or is not currently undergoing telemetry monitoring. Maintaining the telemetry monitoring status may include not adjusting the telemetry monitoring status from currently undergoing telemetry monitoring (e.g., “monitored”) to not currently undergoing telemetry monitoring (e.g., “not monitored”), or vice versa.

By managing the telemetry monitoring status of the patient according to the method 300 as described above, the telemetry management system may determine the telemetry monitoring status for a large number of patients and may store the results of the determination in the patient index. The telemetry management system may then utilize the patient index in order to graphically display the telemetry monitoring status of the patients to care providers, enabling the care providers to more quickly and easily determine desired adjustments to patient care.

Referring now to FIGS. 4A-4B, a flow chart of a method 400 for determining a score for each patient in a patient index based on patient information and telemetry monitoring status via a telemetry management system is shown, according to an exemplary embodiment. In some embodiments, method 400 may be executed by the telemetry management system (e.g., telemetry management system 100 of FIG. 1B) for each patient within the patient index, as indicated at 212 of method 200 of FIG. 2, described above. Although method 400 may be described herein as applied to a single patient within the patient index, it should be understood that the telemetry management system may execute method 400 for each patient within the patient index. Method 400 may be implemented as executable instructions in a stratification module of the telemetry management system, such as the stratification module 118 of FIG. 1B.

At 402, a determination is made of whether a patient is undergoing telemetry monitoring. The determination of whether the patient is undergoing telemetry monitoring may be based on the telemetry monitoring status of the patient, as described above with reference to 210 of method 200 of FIG. 2, and as described with reference to method 300 of FIG. 3. For example, during conditions in which the telemetry monitoring status of a patient is updated while telemetry monitoring data is received for the patient by the telemetry management system (e.g., at 308 or 316 of method 300 shown by FIG. 3 and described above), the telemetry monitoring status of the patient stored in the patient index may indicate that the patient is currently undergoing telemetry monitoring. As a result, the determination may be made at 402 by the telemetry management system that the patient is undergoing telemetry monitoring. However, during conditions in which the telemetry monitoring status of the patient is updated while telemetry monitoring data is not received for the patient by the telemetry management system (e.g., at 306 or 314 of method 300), the telemetry monitoring status of the patient stored in the patient index may indicate that the patient is not currently undergoing telemetry monitoring. As a result, the determination may be made at 402 by the telemetry management system that the patient is not undergoing telemetry monitoring.

If the determination is made that the patient is undergoing telemetry monitoring at 402, the telemetry management system determines at 404 (as shown by FIG. 4A) whether a triggering event (e.g., change in patient condition) has occurred. The triggering event may include a physiological event (e.g., ventricular tachycardia, asystole, etc.) that indicates a possible deterioration in a condition of the patient. In some embodiments, the determination of whether the triggering event has occurred may be based on the patient information acquired by the telemetry management system (e.g., patient records and/or patient lab results, such as indicators of dysrhythmia, heart rate or SpO2 limit violations, etc., acquired by the telemetry management system). As one example, the telemetry management system may perform a comparison of patient information acquired at a time of admittance of a patient (e.g., admittance of the patient to the facility including the patient population managed by the telemetry management system) to patient information acquired at a second, later time following patient admittance (e.g., a time corresponding to acquisition of updated patient information by the telemetry management system, such as a time corresponding to a transmission of patient lab results from a laboratory information system, which may be the LIS 110 shown by FIG. 1B, to the telemetry management system). The telemetry management system may determine if a change in the patient information has occurred between the two times, and if a change has occurred, the telemetry management system may determine whether the condition of the patient has deteriorated. For example, patient lab results may indicate that troponin levels of the patient are in an abnormal range (e.g., a range indicative of heart degradation beyond an expected level corresponding to a diagnosis of the patient). As another example, telemetry monitoring data may indicate QT/QTc prolongation of the patient which may indicate deterioration of the condition of the patient. Deterioration of the condition of the patient may qualify as a triggering event based on pre-determined criteria stored within the memory of the telemetry management system.

In some embodiments, the telemetry management system may compare the patient information at 404 to a previous, most recently acquired patient information in order to determine whether a triggering event has occurred. For example, throughout the duration the patient is treated at the facility, the telemetry management system may periodically acquire updated patient information. At 404 the telemetry management system may compare the most recently updated patient information to an immediately prior version of the patient information in order to determine whether a triggering event has occurred. The criteria for the triggering event may include myocardial infarction, atrial fibrillation, abnormal lab results, diagnosis changes, etc., and may be pre-determined by care providers and stored in the memory of the telemetry management system.

In some embodiments, determining whether a triggering event has occurred at 404 may include notifying the telemetry management system that the triggering event has occurred via updated patient information (e.g., as acquired from the EMR of the patient by the telemetry management system). For example, a clinician may update the EMR of the patient to include a notification that a triggering event has occurred. The telemetry management system may receive the updated EMR and may make the determination at 404 based on the notification stored in the updated EMR. In other embodiments, a clinician may update the patient information directly at the telemetry management system via a user interface of the telemetry management system (e.g., user interface 128 shown by FIG. 1B and described above) in order to indicate that a triggering event has occurred. In other embodiments, the telemetry management system may compare telemetry monitoring data acquired to one or more thresholds in order to determine whether a triggering event has occurred. For example, the telemetry management system may compare a measured QT/QTc in ECG telemetry monitoring data to a threshold QT/QTc in order to determine whether a triggering event has occurred (e.g., during conditions in which the measured QT/QTc exceeds the threshold QT/QTc, the telemetry management system may determine that a triggering event has occurred).

If the determination is made that a triggering event has occurred at 404, patient score is reduced at 406. In some embodiments, as described above, patients may be admitted to the facility with an initial score based on patient diagnosis, an average score of the patient population (e.g., an average score for all patients in the patient index), a pre-determined initial score, etc. The score may be stored in the memory of the telemetry management system and may be associated (e.g., linked) with the patient information stored in the patient index. At 406, the telemetry management system reduces the score of the patient. As one example, the patient may be newly admitted to the facility and the patient score prior to the reduction may correspond to the initial score described above (e.g., the patient score may not yet have been adjusted from the initial score by the telemetry management system). At 406, the telemetry management system may reduce the patient score below the initial score (e.g., the initial score may be 5.0, and the telemetry management system may reduce the score to a number below 5.0, such as 4.0). As another example, a patient that has been admitted to the facility for a duration and has had one or more adjustments to patient score performed by the telemetry management system prior to the determination at 406 may have a score differing from the initial score described above (e.g., resulting from one or more prior executions of the method 400 by the telemetry management system).

The telemetry management system may, at 406, reduce the patient score relative to the most recently determined patient score (e.g., reduce the patient score from 7.0 to 6.0, which in some examples may be an initial or an average score of the patient population. In other embodiments, the telemetry management system may reduce the score by an amount determined via a machine learning model or other deep learning model (e.g., based on training datasets that include patient parameters and known outcomes, as described above).

In some embodiments, the telemetry management system may store the patient score in a data array that is linked with the patient index. In other embodiments, the telemetry management system may append the patient score to the patient index (e.g., store the patient score within a subarray of the patient index). In each embodiment, reducing the patient score at 406 includes adjusting the patient score at the corresponding location in which the patient score is stored in the memory of the telemetry management system.

Reducing the patient score at 406 may include, at 408, setting an alert to increase a duration of telemetry monitoring. Setting the alert to increase the duration of patient telemetry monitoring may include storing the alert in the memory of the telemetry management system. In some embodiments, the alert may include a recommended amount of time by which to increase the duration of telemetry monitoring of the patient.

For example, at the time at which the patient is admitted to the facility, a care provider may place an order to initiate telemetry monitoring of the patient (e.g., the care provider may update the patient information stored within an EMR database, such as an EMR database included within the databases 108 shown by FIG. 1B and described above, to indicate that telemetry monitoring of the patient is desired). The order to initiate telemetry monitoring may include a desired duration for telemetry monitoring, with the desired duration determined by the care provider (e.g., the care provider may determine the desired duration based on pre-determined criteria or guidelines provided by the facility for the diagnosis associated with the patient). At the time that alert is set at 408, initiation of telemetry monitoring of the patient has occurred and the patient may have already been monitored via telemetry monitoring for a portion of the desired duration determined by the care provider (e.g., a portion of the desired duration may have already elapsed). The alert set at 408 may indicate a recommended amount of time by which to extend the duration of the telemetry monitoring of the patient.

In some embodiments, the recommended amount of time indicated by the alert may be based on the remaining duration of telemetry monitoring scheduled for the patient. As one example, the patient may be scheduled to receive telemetry monitoring for a duration (e.g., 24 hours) and may be undergoing the scheduled telemetry monitoring for some amount of time prior to the determination that a triggering event has occurred at 404. As such, when the determination is made at 404, only a portion of the scheduled telemetry monitoring duration may have occurred, with a portion of the scheduled telemetry monitoring duration still remaining. During conditions in which the remaining portion of the scheduled telemetry monitoring duration is larger, such as 20 hours, the telemetry management system may recommend (e.g., via the alert) extending the telemetry monitoring duration by a smaller amount, such as 4 hours. During conditions in which the remaining portion of the scheduled telemetry monitoring duration is smaller, such as 3 hours, the telemetry management system may recommend extending the telemetry monitoring duration by a larger amount, such as 8 hours. In other embodiments, the recommended amount of time may be based on the diagnosis of the patient and/or changes in patient condition (e.g., as determined at 404 and described above). For example, the triggering event determined at 404 may result in a deterioration of the condition of the patient, and the recommended amount of time provided by the alert to extend telemetry monitoring of the patient may be based on a severity of the deterioration and/or severity of the triggering event (e.g., electrolyte imbalance may result in a recommended amount of time of 12 hours, suspected myocardial infarction may result in a recommended amount of time of 24 hours, etc.). The recommended amount of time provided by the alert may be determined according to the telemetry monitoring protocols/standards described above, at least in some examples.

In some embodiments, the telemetry management system may store the alert in a data array that is linked with the data array including the patient index and/or the data array that includes the patient score (e.g., in embodiments in which the patient score is maintained at a separate location within the memory of the telemetry management system relative to the patient index). In other embodiments, the telemetry management system may append the alert to the patient index (e.g., store the alert within a subarray of the patient index). In each embodiment, the alert and patient score are linked to the corresponding patient name within the memory of the telemetry management system such that the telemetry management system may recall each of the patient name, patient score, and alert for display at a display device (e.g., as described further below).

However, if the determination is made that a triggering event has not occurred at 404, a determination is made at 410 of whether an incomplete telemetry monitoring cessation is indicated. As explained above with respect to FIG. 3, a telemetry monitoring cessation may be determined to be incomplete if an order for telemetry monitoring cessation is received by the telemetry management system, but telemetry monitoring data is still received (e.g., from the telemetry monitoring sensors). The indication of incomplete telemetry cessation may be included with the patient telemetry monitoring status stored in the memory of the telemetry management system, as described above with reference to 308 of method 300 shown by FIG. 3. The telemetry management system may reference the patient telemetry monitoring status at 410 in order to perform the determination of whether telemetry monitoring cessation is incomplete.

If incomplete telemetry monitoring cessation is indicated at 410, the patient score is maintained at 412. Maintaining the patient score may include not adjusting the patient score stored in the memory of the telemetry management system (e.g., not increasing or decreasing the patient score).

Further, maintaining the patient score at 412 may include, at 414, setting an alert to remove the patient from telemetry monitoring. In some embodiments, the alert to remove the patient from telemetry monitoring may include a recommendation or reminder to a care provider to perform cessation of telemetry monitoring of the patient (e.g., decouple the patient from telemetry monitoring sensors). For example, because incomplete telemetry monitoring cessation is indicated at 410, a care provider may have previously ordered cessation of telemetry monitoring of the patient but the cessation order may not have yet been executed at the time the determination is made at 410. The alert set at 414 may serve as a reminder to the care provider to remove the patient from telemetry monitoring (e.g., in order to reduce an amount of facility resources allocated toward maintaining telemetry monitoring of the patient, such as clinicians assigned to continuously monitor the condition of the patient via electronic signals output by the telemetry monitoring sensors). The alert set at 414 may be stored in the memory of the telemetry management system and may be scheduled to output along with a stratified patient list generated by the telemetry management system, as described further below.

However, if incomplete telemetry monitoring cessation is not indicated at 410, a determination is made at 416 of whether patient condition is within a telemetry monitoring criteria. In some embodiments, the telemetry monitoring criteria may be a pre-determined criteria provided by care providers and stored in the memory of the telemetry management system (e.g., obtained from the protocols/standards system 111). For example, a lead clinician or lead care provider of the facility may determine a list of patient conditions (e.g., patient diagnoses) for which telemetry monitoring is desired, and the list of patient conditions may be stored within the memory of the telemetry management system as the telemetry monitoring criteria. The telemetry management system may compare the patient condition at 416 to the telemetry monitoring criteria in order to determine whether the patient condition is within or outside of the telemetry monitoring criteria. Patient condition may correspond to a diagnosis of the patient during admittance of the patient to the facility, in some embodiments. The diagnosis of the patient may be stored within the EMR associated with the patient and may be a portion of the patient information acquired by the telemetry management system. As one example, the patient may be admitted to the facility with a diagnosis of respiratory illness and the telemetry management system may compare the patient diagnosis (e.g., respiratory illness) to the pre-determined list of patient diagnoses forming the telemetry monitoring criteria to determine whether the patient diagnosis meets the criteria (e.g., whether the diagnosis of respiratory illness is on the pre-determined list of patient diagnoses). Determining whether the patient condition is within the telemetry monitoring criteria may include determining whether the telemetry monitoring protocols or standards stored at and obtained via protocols/standards system 111 indicate that telemetry monitoring is recommended for the patient condition, at least in some examples.

If the patient condition is within the telemetry monitoring criteria at 416, a determination is made at 418 of whether a telemetry monitoring duration is greater than a first threshold duration. The telemetry monitoring duration may be the total amount of time the patient has been monitored via telemetry monitoring following a telemetry monitoring initiation order for the patient and/or the total amount of time the patient has been monitored via telemetry monitoring following admission of the patient to the facility. For example, the telemetry monitoring duration may correspond to the amount of time elapsed from initiation of telemetry monitoring of the patient (e.g., starting from the time at which the patient is coupled to telemetry monitoring sensors) up to the determination at 418. In some embodiments, the telemetry management system may track the telemetry monitoring duration by comparing a time at which telemetry monitoring data of the patient is transmitted to the telemetry management system to a time at which the determination is made at 418.

The telemetry monitoring duration is compared to the first threshold duration (e.g., by the telemetry management system) in order to determine whether the telemetry monitoring duration is greater than the first threshold duration. In some embodiments, the first threshold duration may be a pre-determined maximum desired telemetry monitoring duration set by the lead clinician or lead care provider of the facility (e.g., 24 hours) for patients that have not had a triggering event occur (as determined at 404) and are within the telemetry monitoring criteria (as determined at 416). In other embodiments, the first threshold duration may be a pre-determined duration associated with the diagnosis assigned to the patient at the time of admittance of the patient to the facility (e.g., for patients admitted to the facility for atrial fibrillation, the first threshold duration may be 12 hours, and for patients admitted to the facility for syncope, the first threshold duration may be 24 hours). As such, the first threshold duration may be a threshold duration that, without any deterioration of the condition of the patient (e.g., due to a triggering event) and with the patient's condition being within the telemetry monitoring criteria, corresponds to a desired maximum amount of time to monitor the patient via telemetry monitoring. The first duration may be determined from the telemetry monitoring protocol or standard for the patient condition.

If the telemetry monitoring duration is greater than the first threshold duration at 416, the patient score is increased at 420. In some embodiments, as described above, patients may be admitted to the facility with an initial score based on patient diagnosis, an average score of the patient population, a pre-determined initial score, etc. The score may be stored in the memory of the telemetry management system and may be associated (e.g., linked) with the patient information stored in the patient index. When the patient has been monitored via the telemetry monitoring for a duration that is longer than the recommended amount for the patient condition, the patient score may be increased. Doing so may adjust the rank of the patient, thereby alerting any care providers that the monitoring status of the patient should be reviewed for cessation. For example, the telemetry management system may increase the patient score above the initial score (e.g., the initial score may be 5.0, and the telemetry management system may increase the score to a number above 5.0, such as 6.0). As another example, a patient that has been admitted to the facility for a duration and has had one or more adjustments to patient score performed by the telemetry management system prior to the determination at 420 may have a score differing from the initial score described above (e.g., resulting from one or more prior executions of the method 400 by the telemetry management system).

The telemetry management system may, at 420, increase the patient score relative to the most recently determined patient score (e.g., increase the patient score from 7.0 to 8.0). In some embodiments, responsive to the determination of the telemetry monitoring duration being greater than the first threshold duration at 418, the telemetry management system may increase the score of the patient above an average score of the patient population at 420 (e.g., above a mean or median score of the patient population). Increasing the patient score in this way may ensure that at least 50% of the patient population has a score lower than the score of the patient. Increasing the score in this way may move the patient's name higher in a stratified patient list generated by the telemetry management system (with an example of the stratified patient list shown by FIG. 6 and described further below). In yet other embodiments, the telemetry management system may increase the patient score at least partially based on the diagnosis of the patient. For example, a patient having a first diagnosis (e.g., syncope) may have their score increased by a larger amount at 420 than a patient having a different, second diagnosis (e.g., myocardial infarction). In some embodiments, the telemetry management system may increase the score by an amount determined via a machine learning model or other deep learning model (e.g., based on training datasets that include patient parameters and known outcomes, as described above). In some examples, the telemetry management system may increase the score by an amount indicated by the telemetry monitoring protocol or standard.

In some embodiments, the telemetry management system may increase the patient score at 420 based on an amount of time by which the telemetry monitoring duration exceeds the first threshold duration (e.g., as determined at 418). For example, a first patient and a second patient may each have a same diagnosis (e.g., syncope), and a telemetry monitoring duration of each patient may exceed the first threshold duration. As one example, the first threshold duration for the diagnosis of syncope may be a pre-determined duration defined by the telemetry monitoring criteria (e.g., 24 hours), and at 418, the first patient may have already been monitored via telemetry monitoring for 25 hours while the second patient may have already been monitored via telemetry monitoring for 27 hours. The telemetry management system may, at 420, increase the patient score of the second patient by an amount such that the rank of the second patient is higher in the stratified list than that of the first patient (e.g., to indicate that removal of the second patient from telemetry monitoring is of higher priority than removal of the first patient from telemetry monitoring).

As described above, in some embodiments, the telemetry management system may store the patient score in a data array that is linked with the data array including the patient index. In other embodiments, the telemetry management system may append the patient score to the patient index (e.g., store the patient score within a subarray of the patient index). In each embodiment, increasing the patient score at 420 includes adjusting the patient score at the corresponding location in which the patient score is stored in the memory of the telemetry management system.

Increasing the patient score at 420 may include, at 422, setting an alert to review patient telemetry monitoring status. In some embodiments, the alert to review patient telemetry monitoring status may include a recommendation or reminder to a care provider to consider providing a telemetry monitoring cessation order for the patient (e.g., request that the patient be decoupled from telemetry monitoring sensors). For example, because the duration of telemetry monitoring of the patient is greater than the first threshold duration, a benefit of further monitoring the patient via telemetry monitoring may be decreased (e.g., a condition of the patient may be relatively stable and not deteriorating, such that the patient may be monitored periodically in person by a nurse or other clinician and not via telemetry monitoring). The alert set at 420 may serve as a reminder to review the condition of the patient in order to determine whether further telemetry monitoring is desired.

However, if the telemetry monitoring duration is less than the first threshold duration at 418, the patient score is maintained at 424. Maintaining the patient score may include not adjusting the patient score stored in the memory of the telemetry management system (e.g., not increasing or decreasing the patient score).

Returning to 416, if the patient condition is not within the telemetry monitoring criteria, a determination is made at 426 of whether the telemetry monitoring duration is greater than a second threshold duration. For example, a patient may be undergoing telemetry monitoring despite the patient condition being outside of the telemetry monitoring criteria if a clinician orders telemetry monitoring of the patient to assess whether the patient has a suspected, but undiagnosed, condition. The telemetry monitoring duration is compared to the second threshold duration (e.g., by the telemetry management system) in order to determine whether the telemetry monitoring duration is greater than the second threshold duration. In some embodiments, the second threshold duration may be a pre-determined maximum desired telemetry monitoring duration set by the lead clinician or lead care provider of the facility (e.g., 12 hours) for patients that have not had a triggering event occur (as determined at 404) and are not within the telemetry monitoring criteria (as determined at 416). Further, in embodiments in which both of the first threshold duration (described above with reference to 418) and second threshold duration are pre-determined and set by the lead clinician or lead care provider (or other staff of the facility), the second threshold duration may be a smaller amount of time than the first threshold duration.

The second threshold duration may be a threshold duration that, without any deterioration of the condition of the patient (e.g., due to a triggering event) and with the patient's condition being outside of the telemetry monitoring criteria, corresponds to a desired maximum amount of time to monitor the patient via telemetry monitoring. For example, despite the patient condition being outside of the telemetry monitoring criteria, a clinician may order telemetry monitoring of the patient in order to assess whether the patient has a suspected, but undiagnosed, condition. In such cases, it may be desirable to reduce prolonged monitoring of the patient via telemetry monitoring in order to reduce an amount of facility resources allocated toward maintaining telemetry monitoring of the patient. Comparing the telemetry monitoring duration to the second threshold duration at 426 may enable the telemetry management system to identify patients that are less likely to benefit from additional telemetry monitoring, as described below.

If the telemetry monitoring duration is greater than the second threshold duration at 426, the patient score is increased at 428. Increasing the patient score at 428 may be similar to increasing the patient score at 420, as described above. In some embodiments, the patient score may be increased at 428 by a greater amount relative to the increase in patient score described above with reference to 420. For example, the increase in patient score at 420 may be a first pre-determined amount (e.g., an increase of 1.0), and the increase in patient score at 428 may be a greater, second pre-determined amount (e.g., an increase of 2.0). As another example, as described above, the increase in patient score at 420 may be at least partially based on the diagnosis of the patient, and similarly, the increase in patient score at 426 may be at least partially based on the diagnosis of the patient. An average (e.g., mean or median) increase in patient score at 426 (e.g., for all patient conditions outside of the telemetry monitoring criteria) may be greater than an average increase in patient score at 420 (e.g., for all patient conditions within the telemetry monitoring criteria), such that patients having a condition outside of the telemetry monitoring criteria may often have an adjusted patient score that is higher than patients having a condition within the telemetry monitoring criteria (and may, on average, be positioned at a higher ranking within a stratified patient list generated by the telemetry management system). In some embodiments, the telemetry management system may increase the score by an amount determined via a machine learning model or other deep learning model (e.g., based on training datasets that include patient parameters and known outcomes, as described above).

In some embodiments, the telemetry management system may increase the patient score at 428 based on an amount of time by which the telemetry monitoring duration exceeds the second threshold duration (e.g., as determined at 426). For example, a first patient and a second patient may each have a same diagnosis (e.g., UTI), and a telemetry monitoring duration of each patient may exceed the second threshold duration. As one example, the second threshold duration may be a pre-determined duration used in situations in which the patient condition (e.g., diagnosis) is not within the telemetry monitoring criteria (e.g., 12 hours, as one non-limiting example). At 428, the first patient may have already been monitored via telemetry monitoring for 13 hours while the second patient may have already been monitored via telemetry monitoring for 15 hours. The telemetry management system may, at 430, increase the patient score of the second patient by an amount such that the rank of the second patient is higher in the stratified list than that of the first patient (e.g., to indicate that removal of the second patient from telemetry monitoring is of higher priority than removal of the first patient from telemetry monitoring).

Increasing the patient score at 428 may include, at 430, setting an alert to review patient telemetry monitoring status. The alert to review patient telemetry monitoring status may be similar to the alerts described above with reference to 422.

However, if the telemetry monitoring duration is not greater than the second threshold duration at 426, the patient score is maintained at 432. Maintaining the patient score may include not adjusting the patient score stored in the memory of the telemetry management system (e.g., not increasing or decreasing the patient score).

Although the patient score is described as increasing at 420 and 428 responsive to the telemetry monitoring duration exceeding a threshold duration (e.g., the first threshold duration and second threshold duration, respectively), in some embodiments the patient score may increase responsive to other determinations. For example, a patient may be admitted with a diagnosis that may be associated with an expected duration of treatment for recovery. However, if the recovery of the patient occurs more quickly than anticipated (e.g., as determined based on the telemetry monitoring data, lab results, and/or updates to the patient information provided by a care provider), the telemetry management system may increase the score of the patient. In another example, an initial diagnosis of a patient provided by a care provider may be updated to a different, less severe diagnosis during treatment of the patient. The telemetry management system may acquire the updated diagnosis and may increase the score of the patient accordingly (e.g., increase the score based on the updated diagnosis, with the increased score corresponding to a score associated with the updated diagnosis). In some embodiments, the telemetry management system may utilize a machine learning model or other deep learning model in order to determine whether the recovery rate of the patient warrants increasing the score of the patient (e.g., based on previous outcomes for patients having a similar diagnosis).

Returning now to 402 of FIG. 4A, if the patient is not undergoing telemetry monitoring at 402, a determination is made at 434 (shown by FIG. 4B) of whether a triggering event has occurred. The determination of whether a triggering event has occurred at 434 may be similar to the determination made at 404, shown by FIG. 4A and described above. The triggering event may indicate that the patient, who was not previously monitored via telemetry monitoring, should now be monitored via telemetry monitoring. The triggering event may be detected automatically by the telemetry management system (e.g., from a lab test result, medication prescription, diagnosis change, or order from a care provider).

If it is determined that a triggering event has occurred at 434, patient score is reduced at 436. Reducing the patient score at 436 may be similar to reducing the patient score at 406, described above. For example, the telemetry management system may, at 436, reduce the patient score relative to the most recently determined patient score (e.g., for the same patient). In some embodiments, the telemetry management system may reduce the score by an amount determined via a machine learning model or other deep learning model (e.g., based on training datasets that include patient parameters and known outcomes, as described above).

Reducing the patient score at 436 may include, at 438, setting an alert to add the patient to telemetry monitoring. In some embodiments, the alert to add the patient to telemetry monitoring may include a recommendation or reminder to a care provider to perform initiation of telemetry monitoring of the patient (e.g., couple the patient to telemetry monitoring sensors). For example, due to the occurrence of the triggering event (e.g., as determined at 434), the condition of the patient may have an increased likelihood of deterioration. It may be desirable to initiate telemetry monitoring of the patient in order to monitor the condition of the patient for deterioration. The alert set at 438 may serve as a reminder to the care provider to add the patient to telemetry monitoring.

However, if it is determined at 434 that a triggering event has not occurred, a determination is made at 440 of whether incomplete telemetry monitoring initiation is indicated. The indication of incomplete telemetry initiation may be included with the patient telemetry monitoring status stored in the memory of the telemetry management system, as described above with reference to 314 of method 300 shown by FIG. 3. The telemetry management system may reference the patient telemetry monitoring status at 440 in order to perform the determination of whether telemetry monitoring cessation is incomplete.

If incomplete telemetry monitoring initiation is indicated at 440, patient score is maintained at 442. Maintaining the patient score may include not adjusting the patient score stored in the memory of the telemetry management system (e.g., not increasing or decreasing the patient score).

However, if incomplete telemetry monitoring initiation is not indicated at 440, a determination is made at 446 of whether the patient condition is within telemetry monitoring criteria. The determination at 446 may be similar to the determination at 416, shown by FIG. 4A and described above.

If it is determined that the patient conditions is not within telemetry monitoring criteria at 446, the patient score is maintained at 448. Maintaining the patient score may include not adjusting the patient score stored in the memory of the telemetry management system (e.g., not increasing or decreasing the patient score). In some embodiments, maintaining the score at 448 may further include displaying a recommendation to add telemetry monitoring of the patient, based on the patient score (e.g., for lower scores, addition of telemetry monitoring of the patient may be recommended even when the patient is not within telemetry monitoring criteria at 446 and is not currently being monitored via telemetry monitoring as determined at 402).

However, if it is determined that the patient condition is within telemetry monitoring criteria at 446, the patient score is reduced at 450. In some embodiments, reducing the patient score at 450 may be similar to reducing the patient score at 436 as described above. However, in other embodiments, the reduction of patient score at 450 may be less (e.g., less of a reduction) than the reduction of patient score at 436. For example, the triggering event (as determined at 434) leading to the reduction in patient score at 436 may be indicative of a deterioration in patient condition. Although the condition of the patient is within the telemetry monitoring criteria as determined at 446, the patient condition may not be deteriorating and may be relatively stable compared to patients that have had a triggering event occur. As such, the reduction in score at 450 may be less of a reduction than the reduction described above at 436 (e.g., such that patients that have had a triggering event occur may have a lower ranking in a stratified patient list generated by the telemetry management system relative to patients that have not had a triggering event occur but have a condition within the telemetry monitoring criteria). In some embodiments, the telemetry management system may reduce the score by an amount determined via a machine learning model or other deep learning model (e.g., based on training datasets that include patient parameters and known outcomes, as described above).

Reducing the patient score at 450 may include, at 452, setting an alert to review patient telemetry monitoring status. In some embodiments, the alert to review patient telemetry monitoring status may include a recommendation or reminder to a care provider to consider providing a telemetry monitoring initiation order for the patient (e.g., request that the patient be coupled to telemetry monitoring sensors). For example, because the patient condition is within the telemetry monitoring criteria, the patient may benefit from telemetry monitoring (e.g., a condition of the patient may be of sufficient severity to warrant telemetry monitoring). The alert set at 452 may serve as a reminder to review the condition of the patient in order to determine whether to monitor the patient via telemetry monitoring.

Referring now to FIGS. 5-6, block diagrams are shown schematically illustrating information acquisition and patient stratification by the telemetry management system 100 of FIG. 1B. Specifically, FIG. 5 schematically illustrates acquisition of a patient index including a list of patients and patient information by the telemetry management system of FIG. 1B, and FIG. 6 schematically illustrates forming a stratified patient list based on a score of each patient via the telemetry management system of FIG. 1B and displaying the stratified patient list at a display device of the telemetry management system. In some embodiments, the telemetry management system 100 may execute the methods described above with reference to FIGS. 2-4B in order to perform the information acquisition and patient stratification as illustrated schematically by FIGS. 5-6.

Turning first to FIG. 5, several features shown by FIG. 1B and described above are schematically shown in order to illustrate information acquisition by the telemetry management system 100. The telemetry management system 100 receives information (e.g., data) transmitted through the network 112 from different sources, such as the telemetry monitoring sensors 102, EMR database 108, pharmacy information system 109, and LIS 110. FIG. 5 schematically illustrates a plurality of patients 500 coupled to the telemetry monitoring sensors 102, with the telemetry monitoring sensors 102 configured to transmit telemetry monitoring data 502 to the telemetry management system 100 via network 112. For example, Patient Y is shown coupled to ECG sensor 540 and SpO2 sensor 542, with the ECG sensor 540 configured to transmit ECG telemetry monitoring data 536 to the telemetry management system 100 and SpO2 sensor 542 configured to transmit SpO2 telemetry monitoring data 538 to the telemetry management system 100. The ECG telemetry monitoring data 536 and SpO2 telemetry monitoring data 538 is shown schematically by FIG. 5 for illustrative purposes. Further, telemetry management system 100 receives telemetry monitoring data 502 from each telemetry monitoring sensors 102 coupled to the patients 500. The patients 500 may be a portion of a patient population of a treatment facility (e.g., hospital), and the treatment facility may include additional patients that are not monitored via telemetry monitoring (e.g., patients that do not have telemetry monitoring sensors 102 coupled to them).

The telemetry management system 100 additionally receives data (e.g., patient information) from the systems and databases, such as EMR database 108. FIG. 5 schematically shows patient records 504 as an array including a plurality of sub-arrays. Specifically, the patient records 504 include a list of patient names 510 (e.g., patient list) illustrated as a first sub-array, patient diagnoses/indications 512 illustrated as a second sub-array, telemetry monitoring orders 514 illustrated as a third sub-array, and patient events 516 illustrated as a fourth sub-array. In some embodiments, the record of patient events 516 includes records of triggering events that have occurred. The determination of whether a triggering event has occurred at 404 and 434 of method 400 shown by FIGS. 4A-4B may be based at least in part on the data stored in the patient events 516 portion of the patient records 504 (e.g., if the patient events 516 portion includes data indicating a triggering event associated with a corresponding patient in the patient list 510, the telemetry management system may determine that the triggering event has occurred for that patient at 404 or 434). The patient records 504 are illustrated as being transmitted from EMR database 108 to telemetry management system 100 via network 112, although the patient information may be obtained from alternative or additional systems and databases.

The telemetry management system 100 may receive additional patient information in the form of lab results 506 from LIS 110. FIG. 5 schematically shows lab results 506 as an array including a plurality of sub-arrays, similar to patient records 504. The patient records 504 and lab results 506 may be referred to herein collectively as patient information. In some embodiments, the patient information (e.g., patient records 504 and lab results 506) is the patient information acquired at 202 of method 400 (e.g., the patient records 504 are the patient records acquired at 204 of method 400, and the lab results 506 are the lab results acquired at 206 of method 400). The lab results 506 include a list of patient names 518 illustrated as a first sub-array and a results summary 520 illustrated as a second sub-array. The results summary 520 includes a list of lab results for each patient in the patient list 518. The lab results 506 are illustrated as being transmitted from LIS 110 to telemetry management system 100 via network 112. The telemetry management system may compare the patient list 510 of the patient records 504 with the patient list 506 of the lab results in order to link (e.g., associate) the results summary 520 for patients within the patient list 506 with the corresponding patients in the patient list 510 of the patient records 504. Specifically, the telemetry management system may combine the results summary 520 with the patient records 504, as described in further detail below.

The telemetry management system 100 generates (e.g., compiles) patient index 508 and stores the patient index 508 in memory 116, as illustrated schematically by FIG. 5. In some embodiments, the patient index 508 is the patient index described above with reference to method 200 shown by FIG. 2 (e.g., the patient index 508 is a non-limiting example of the patient index generated according to the method of FIG. 2). In the embodiment shown by FIG. 5, generating the patient index 508 includes compiling the patient index 508 by combining the data received by the telemetry management system 100 from the telemetry monitoring sensors 102 (e.g., telemetry monitoring data 502), EMR database 108 and/or other systems and databases (e.g., patient records 504), and LIS 110 (e.g., lab results 506). By combining the data from the different sources described above, the patient index 508 may include a wide variety of patient information and may be utilized by the telemetry management system 100 to determine a score for each patient and form a stratified patient list, as described above with reference to method 200 of FIG. 2.

The patient index 508 shown schematically by FIG. 5 includes a list of patient names 522, diagnoses/indications 524, ECG telemetry monitoring data 526, SpO2 telemetry monitoring data 528, telemetry monitoring orders 514, telemetry monitoring duration ordered 530, telemetry monitoring duration elapsed 532, results summary 534, and events recorded 544. In some embodiments, the telemetry management system 100 may generate the telemetry monitoring duration elapsed 532 for each patient based on a difference between a time at which telemetry monitoring data (e.g., ECG telemetry monitoring data 526 and/or SpO2 telemetry monitoring data 528) is initially received for the patient (e.g., a time at which telemetry monitoring data is first received) and a time at which the patient index 508 is generated (e.g., compiled by the telemetry management system 100). In other embodiments, the telemetry management system 100 may continuously track (e.g., measure) a current time and may compare the tracked time to the time at which telemetry data is first received for each patient in order to determine the telemetry monitoring duration elapsed 532 for each patient. The telemetry monitoring duration elapsed corresponds to a telemetry monitoring duration for each patient (e.g., an amount of time each patient has been monitored via telemetry monitoring).

The lists of patient names (e.g., 510, 518, and 522) may each be the same in some embodiments and may be utilized by the telemetry management system 100 in order to link patient information (e.g., diagnosis from diagnoses 512, lab results from results summary 520, telemetry monitoring data from telemetry monitoring sensors 102, etc.) from the different data sources with the corresponding patient in the patient index 508. Further, diagnoses/indications 524 may be the same as diagnoses/indications 512, telemetry monitoring orders 514 may be the same as telemetry monitoring orders 530, results summary 534 may be the same as results summary 520, and patient events 516 may be the same as events 544 (e.g., in some embodiments, the diagnoses 512, telemetry monitoring orders 514, results summary 520, and patient events 516 may be stored directly in the patient index 508 without modification, such that the diagnoses 524, telemetry monitoring orders 530, results summary 534, and events 544 include the same data as diagnoses 512, telemetry monitoring orders 514, results summary 520, and patient events 516, respectively).

The patient index 508 may be input to the stratification module 118 of the telemetry management system 100. The stratification module 118 may interface with processors 120 in order to determine a score for each patient in the patient index 508 based on the patient information stored within the patient index 508 and the telemetry monitoring status of each patient. The determination of the score for each patient as performed by the telemetry management system may be the same as the examples described above with reference to method 400 illustrated by the flowchart of FIGS. 4A-4B. In some embodiments, the telemetry monitoring status of each patient may be determined by the telemetry management system 100 based on the patient telemetry monitoring data (e.g., ECG telemetry monitoring data 526 and/or SpO2 telemetry monitoring data 528), telemetry monitoring orders 530, and/or telemetry monitoring duration elapsed 532 and may be stored in the memory 116 of the telemetry management system 100. In some embodiments, in addition to the duration of telemetry monitoring ordered, the telemetry monitoring orders 530 may include orders by the care providers to initiate telemetry monitoring of a patient or to cease telemetry monitoring of a patient. The telemetry monitoring status of each patient may include an indication of whether the patient is currently monitored via telemetry monitoring (e.g., as determined by whether telemetry monitoring data is currently received for the patient by the telemetry management system, the same as the examples described above with reference to method 300 shown by the flowchart of FIG. 3), and the telemetry monitoring status may further include an indication of whether an order to initiate telemetry monitoring or cease telemetry monitoring has been made for the patient. The patient scores and telemetry monitoring status may each be stored in the memory 116 of the telemetry management system 100.

The stratification module 118 may interface with the processors 120 in order to form a stratified patient list 600 based on the score of each patient, and display the stratified patient list 600 at the display device 126. Further, the telemetry management system may determine alerts for patients within the stratified patient list 600 (e.g., the same as the examples described above with reference to method 400 illustrated by the flowchart of FIGS. 4A-4B), and may display the alerts and telemetry monitoring status of the patients with the stratified patient list 600. In the embodiment shown by FIG. 6, for example, the stratified patient list 600 includes stratified patient names 602, patient scores 604, patient ranking 606, telemetry monitoring status 608, and alerts 610. Although the stratified patient list 600 is shown as one illustrative example of the display of the stratified patient list 600 at the display device 126, in other embodiments the stratified patient list may be displayed in a different way (e.g., the display device 126 may display only the stratified patient names 803, telemetry monitoring status 608, and alerts 610, without displaying the scores 604 and/or ranking 606).

FIG. 7 shows another example of a patient list 708 stored in memory 116 of the telemetry management system 100. The patient list 708 illustrated in FIG. 7 includes a patient name sub-array 722, a diagnosis/indication sub-array 724, a duration of monitoring ordered sub-array 730, a duration of monitoring elapsed sub-array 732, and a results sub-array 734, similar to the sub-arrays described above with respect to FIGS. 5 and 6. Further, the patient list 708 includes an events sub-array 733 and a medication sub-array 735. Accordingly, for each patient included in the patient list, a diagnosis, amount of monitored ordered, amount of monitoring that has elapsed, any triggering events, medications, and results are stored in the patient list.

The patient list may be stratified according to a patient score assigned to each patient, as explained above. Once the patient scores are assigned, telemetry monitoring status for each patient is determined, and any indicated alarms are generated. Thus, as shown, a stratified patient list 750, which is displayed on display device 126, is generated from patient list 708 and includes patient name 752, patient score 754, patient rank 756, telemetry monitoring status 758, and alerts 760.

The patient list and subsequent stratified list shown in FIG. 7 includes patients that are currently undergoing monitoring (patients A, C, D, E, and F) as well as patients that are not currently undergoing monitoring (patient B). Patient B is not undergoing monitoring because the diagnosis/indication for patient B (respiratory illness) does not automatically dictate monitoring. However, as indicated at the medications sub-array 735, patient B received an order to start taking levofloxacin (e.g., to treat the respiratory illness), which may cause QT prolongation. Thus, as appreciated by the stratified list 750, the score for patient B may be lowered relative to the score assigned to patient B at admittance (e.g., from 10.0 to 5.0) due to the administration of levofloxacin, which may cause patient B to have the lowest rank of the patients shown in the stratified list 750. Further, an alert may be output for patient B indicating that patient B should be monitored.

In contrast, patient A may be ranked the highest of the patients shown in the stratified list 750, with a score of 10.0. This is due to patient A having been monitored for 30 hours, while the protocol for the diagnosis/indication for patient A (e.g., syncope) only recommends monitoring for 24 hours. Thus, in addition to the high score and ranking for patient A, an alert is output indicating that the monitoring status for patient A should be reviewed. Likewise, an alert may be output for patient D, ranked second, due to patient D being monitored yet having a diagnosis/indication (e.g., UTI) that does not require monitoring, and with no triggering events or adverse lab test results. The alert may indicate that the monitoring status for patient D should be reviewed, in order to determine if continued monitoring of patient D is necessary. Patients E and F may be within the monitoring protocol for the respective conditions (e.g., monitoring to rule out myocardial infarction and monitoring for syncope), and thus no alerts are output for patients E and F. Patient C has been monitored for a duration longer than recommended for the diagnosis/indication (e.g., ruling out MI), but patient C received a lab result (abnormal cardiac biomarkers) indicating that continued monitoring is warranted, and thus no alert is output for patient C.

While the embodiments disclosed herein include a stratified list being generated and displayed to one or more care providers, other mechanisms for conveying the patient score, patient rank, and/or telemetry monitoring status for each patient are possible without departing from the scope of this disclosure. For example, rather than generating a stratified list that includes the patients ranked in ascending or descending order based on patient score, the patients may be placed into bins based on patient score. For example, the patients may be placed into one of three bins, a first bin for patients having a score indicating that telemetry monitoring cessation may be considered, a second bind for patients having a score indicating that current telemetry monitoring status be continued (whether the patients are currently being monitored or not being monitored), and a third bin for patients having a score indicating that telemetry monitoring initiation may be considered. The bins may be displayed as separate lists of patients, with or without the associated patient score. Further, the stratified list of patients described herein, or the different bins of patients described above, may be updated continuously (e.g., every minute or two) or intermittently (e.g., every hour, every 12 hours, etc.). The stratified list or bins may be displayed continuously, only when requested, and/or only once updates to the list or bins have been made.

FIG. 8A shows an example timeline 800 of determined patient scores for two example patients over a telemetry monitoring duration. Timeline 800 includes time plotted on the x-axis and patient score plotted on the y-axis. Plots for two patients are shown by curve 802 (for a first patient) and curve 804 (for a second patient). The patient score may indicate a relative need for telemetry monitoring where a higher score indicates less of a need for telemetry monitoring and a lower score indicates more of a need for telemetry monitoring. For illustrative clarification, FIG. 8B shows timeline 800 with plot 802 and without plot 804, while FIG. 8C shows timeline 800 with plot 804 and without plot 802. FIGS. 8A-8C are referred to collectively below.

At time t1, the first and second patients are each assigned an initial patient score based on a respective condition or indication the patients presented with at time of admittance to the medical facility. The first patient presented with a urinary tract infection (UTI), and thus may be given a high initial patient score (e.g., a score of 10 as shown), as no telemetry monitoring is indicated for patients having UTIs. The second patient presented with symptoms that are not definitively associated with a diagnosis, but potentially indicative of myocardial infarction. Thus, at time t1, the second patient is assigned a lower patient score (e.g., of 5 as shown). An order to initiate telemetry monitoring is entered for the second patient in order to rule out MI.

The respective patient scores are maintained between time t1 and t2. The first patient is not monitored between time t1 and time t2, while the second patient is monitored between time t1 and time t2. At time t2, the first patient commences antibiotic treatment of the UTI with levofloxacin, which may cause QT prolongation. As such, the patient score for the first patient drops at time t2 to 5. An alert may be output notifying the care providers that telemetry monitoring for the first patient is recommended, and thus telemetry monitoring for the first patient is initiated. The patient scores for both the first and second patients remain at 5 until time t3.

At time t3, the patient scores for both patients increase. For the first patient, the patient score increases by a first amount (e.g., to 6), as the first patient has been monitored for a specified duration (e.g., 24 hours, between time t2 and t3) without showing QT prolongation issues. For the second patient, the patient score increases by a second amount (e.g., to 7.5), as the second patient has been monitored for a specified duration (e.g., 48 hours, between time t1 and t3) without any events, medications, or lab test results indicating additional monitoring is needed. Because the two patients have different conditions, the scores may increase by different amounts.

At time t4, a lab test result for the second patient is received at the telemetry management system, indicating abnormal cardiac biomarkers. Thus, the patient score for the second patient drops at time t4 (e.g., to 4.5). Accordingly, even though the second patient was previously a candidate for telemetry monitoring cessation, the abnormal test results cause the patient score to drop and the second patient continues to be monitored. Likewise, while the first patient has undergone monitoring for a sufficient amount of time without any mitigating events, test results, etc., the first patient continues to be monitored. For example, the patient score may not have been raised high enough to cause the telemetry management system to output an alert to consider telemetry monitoring cessation, or the care providers may have decided to allow monitoring to continue due to other reasons (e.g., patient described symptoms, care provider preference).

At time t5, the first patient has been monitored for another period of time (e.g., another 24 hours) without events, results, or medications indicating monitoring is still needed. Thus, the patient score for the first patient is increased again (e.g., to 10), and an alert is output notifying the care providers to review the monitoring status of the first patient and consider cessation of monitoring. The first patient may then subsequently be removed from monitoring. The second patient continues to be monitored with the patient score of 4.5. At time t6, the patient score for the second patient is increased (e.g., to 7.5), as the second patient has been monitored for a recommended duration (e.g., 48 hours). The care providers may then choose to end the monitoring for the second patient.

Referring now to FIG. 8D, an example timeline 806 is shown of determined patient score for a third example patient over a telemetry monitoring duration. Timeline 806 includes time plotted on the x-axis and patient score plotted on the y-axis. The plot for the patient is shown by curve 808. The patient score may indicate a relative need for telemetry monitoring, where a higher score indicates less of a need for telemetry monitoring and a lower score indicates more of a need for telemetry monitoring.

At time t1, the patient is assigned an initial patient score based on a respective condition or indication the patient presented with at time of admittance to the medical facility. The patient presented with symptoms that confirm a diagnosis of myocardial infarction. Thus, the patient is assigned a patient score near a lower end of a score range (e.g., a patient score of 2.5 as shown). An order to initiate telemetry monitoring is entered for the patient in order to monitor patient recovery.

The patient score is maintained between time t1 and t2, and the patient is monitored via telemetry monitoring between time t1 and time t2. At time t2, the telemetry management system determines that no triggering events have occurred between time t1 and time t2. As a result, the patient score increases by a first amount at time t2 (e.g., to 5), as the patient has been monitored for a specified duration (e.g., 12 hours) without showing signs of degradation of patient condition.

At time t3, the patient has undergone monitoring for an additional duration (e.g., an additional 12 hours, such that the overall time the patient has been monitored since time t1 is 24 hours). At time t3, the telemetry management system determines that no triggering events have occurred between time t2 and time t3. As a result, the patient score increases by a second amount at time t3 (e.g., to 7.5), as the patient has been monitored for a specified duration (e.g., 24 hours) without showing signs of degradation of patient condition.

At time t4, the patient has undergone monitoring for an additional duration (e.g., an additional 24 hours, such that the overall time the patient has been monitored since time t1 is 48 hours). At time t4, the telemetry management system determines that no triggering events have occurred between time t3 and time t4. Further, at time t4, the total telemetry monitoring duration of the patient since time t1 may exceed a duration for monitoring patients having the diagnosis of myocardial infarction according to pre-determined patient care guidelines established by the monitoring facility (e.g., hospital). As a result, the patient score increases by a third amount at time t4 (e.g., to 10), and an alert is output notifying the care providers to review the monitoring status of the patient and consider cessation of monitoring (e.g., as indicated by the solid line portion of curve 808, where the dashed line portion of curve 808 indicates time prior to the output of the alert). The patient may then subsequently be removed from monitoring.

Although the embodiments described herein refer to ranking patients by patient score, with higher patient scores assigned to patients that may benefit less from telemetry monitoring, with lower patient scores assigned to patients that may benefit more from telemetry monitoring, and with the patients ranked from highest score to lowest score in the stratified patient list, in other embodiments the ranking of patients may occur differently. For example, in some embodiments, the patients may be ranked from lowest patient score to highest patient score (e.g., with patients having higher patient scores positioned at the bottom of the stratified patient list, and with patients having lower patient scores positioned at the top of the stratified patient list). In other embodiments, the patients in the stratified patient list may be classified according to pre-determined patient score ranges. For example, patients having a patient score between 0.0-3.3 may be classified as low priority for telemetry cessation, patients having a patient score between 3.4-6.6 may be classified as medium priority for telemetry cessation, and patients having a patient score between 6.7-10.0 may be classified as high priority for telemetry cessation. Although the patient scores are described as being within a range of 0.0-10.0 in the embodiments described above, the range is non-limiting and in other embodiments the range may be different (e.g., −10.0 to +10.0, 0.0 to 100.0, etc.). In yet other embodiments, higher patient scores may be assigned to patients that may benefit more from telemetry monitoring, and lower patient scores may be assigned to patients that may benefit less from telemetry monitoring. In such embodiments, increases and decreases to patient score according to method 400 may be reversed. For example, instead of reducing patient score at 406, the patient score may be increased, and instead of increasing patient score at 420 or 428, the patient score may be decreased.

In yet other embodiments, patients may be classified within the stratified patient list according to whether removal of patient telemetry monitoring is recommended, continuation of patient telemetry monitoring is recommended, or initiation of patient telemetry monitoring is recommended, with the recommendations being based on the patient score (e.g., with patients having a patient score within a first range being recommended for removal from telemetry monitoring, patients having a patient score within a second range being recommended for continuation of telemetry monitoring, and patients having a patient score within a third range being recommended for initiation of telemetry monitoring). The above examples are non-limiting, and in other embodiments the ranking of the patients within the stratified patient list may occur in a different way.

In some embodiments, a graphical user interface (GUI) displayed at a display device of the telemetry management system, such as the display device 126 shown by FIG. 1B and described above, may be configured to display a stratified patient list output to the display device by a stratification module of the telemetry management system, such as stratification module 118 shown by FIG. 1B and described above.

The GUI may include one or more sections or portions, such as a first section, a second section, and a third section. The first section may be configured to display patient information for patients that the telemetry management system has determined should be monitored via telemetry monitoring, but are not yet monitored via telemetry monitoring. As one example, the patient information displayed in the first section may be associated with patients for which the telemetry management system has assigned an alert to review patient telemetry monitoring status (e.g., similar to the example described above at 452 of method 400) or an alert to add the patient to telemetry monitoring (e.g., similar to the example described above at 438 of method 400). For each patient having patient information displayed in the first section, the respective patient information may be displayed in a virtual card. Each card in the first section may correspond to a different patient within the facility and may display the respective patient information of the patient. Each card in the first section may include an indicator. In some embodiments, the indicators may include the patient score of the respective patient (e.g., the score assigned to the respective patient by the telemetry management system). Further, in some embodiments, each indicator in the first section may be assigned a first color.

The second section may be configured to display patient information for patients that are being currently monitored via telemetry monitoring but have been recommended to be disconnected from telemetry monitoring by the telemetry management system. As one example, the patient information displayed in the second section may be associated with patients for which the telemetry management system has assigned an alert to remove the patient from telemetry monitoring (e.g., similar to the example described above at 414 of method 400). For each patient having patient information displayed in the second section, the respective patient information may be displayed in a virtual card. Each card may include a respective indicator (with the indicators including patient score in some embodiments). The indicators of the second section may be similar to the indicators of the first section but may be displayed in a different, second color.

The third section may be configured to display patient information for patients that are being currently monitored via telemetry monitoring and have not been recommended to be disconnected from telemetry monitoring by the telemetry management system. As one example, the patient information displayed in the second section may be associated with patients for which the telemetry management system has determined should remain monitored via telemetry monitoring (e.g., similar to the example described above at 424 and/or 432 of method 400). For each patient having patient information displayed in the third section, the respective patient information may be displayed in a virtual card. Each card may include a respective indicator. The indicators of the third section may be similar to the indicators of the first section and/or second section but may be displayed in a different, third color.

In some embodiments, the telemetry management system may categorize or classify patients based on patient information. For example, the patient information may include diagnosis, patient labs, patient prescriptions, cardiology management system outputs, patient events, patient alert history (e.g., alerts from one or more telemetry monitoring devices), etc. The telemetry management system may use the patient information in order to assign patients to pre-determined categories. In some embodiments, the categories may be grouped into parent groupings (e.g., add patient to telemetry monitoring, remove patient from telemetry monitoring, and continue patient telemetry monitoring), and each parent grouping may be assigned a different identification color. Patients within each parent grouping may be further ranked and/or ordered by the telemetry management system in the stratified patient list, and the groupings may be displayed within the GUI. Based on the patient information, patients may be categorized according to any of the following categories: “requires hookup to monitor,” “review order before connecting or hookup with an X time review reminder” (where X is an amount of time, such as 4 hours), “need physician order and connect,” “no action; no monitor needed,” “need physician order,” “remove monitoring,” “continue monitoring,” “need D/C order—non-condition,” or “need D/C order—time met.” Further, some categories may include a physician override option, such as the “review order before connecting or hookup with an X time review reminder,” “need physician order and connect,” “need D/C order—non-condition,” and/or “need D/C order—time met” categories.

As an example of the categorization of the patients, the telemetry management system may first determine whether a given patient is connected to a monitor (e.g., a telemetry monitoring sensor, such as the telemetry monitoring sensors described above). If the patient is not connected to the monitor, a physician order for telemetry monitoring has not been made, and the patient meets guidelines to be unmonitored, the patient may be categorized in the “no action; no monitor needed” category. If the patient is not connected to the monitor, a physician order for telemetry monitoring has not been made, and the patient does not meet guidelines to be unmonitored, the patient is categorized in the “need physician order and connect” category. If the patient is not connected to the monitor, a physician order for telemetry monitoring has been made, and the patient meets guidelines for monitoring, the patient is categorized in the “requires hookup to monitor” category. If the patient is not connected to the monitor, a physician order for telemetry monitoring has been made, and the patient does not meet guidelines for monitoring, the patient is categorized in the “review order before connecting or hookup with an X time review reminder” category.

In some examples, if the patient is connected to a monitor, a physician order for telemetry monitoring has not been made, and the patient does not meet guidelines for monitoring, the patient is categorized in the “remove monitoring” category. If the patient is connected to a monitor, a physician order for telemetry monitoring has not been made, and the patient meets guidelines for monitoring, the patient is categorized in the “need physician order” category. If the patient is connected to a monitor, a physician order for telemetry monitoring has been made, and the patient meets guidelines for monitoring, the patient is categorized in the “continue monitoring” category. If the patient is connected to a monitor, a physician order for telemetry monitoring has been made, the patient does not meet guidelines for monitoring, and the patient meets guidelines for time expiration, the patient is categorized in the “need D/C order—time met” category. If the patient is connected to a monitor, a physician order for telemetry monitoring has been made, the patient does not meet guidelines for monitoring, and the patient does not meet guidelines for time expiration, the patient is categorized in the “need D/C order—non-condition” category.

The above categories are provided as non-limiting examples, and in other embodiments the telemetry management system may categorize the patients into different categories relative to those described above.

In some embodiments, the telemetry management system may display the stratified patient list with other information included. For example, the stratified patient list may include, for each patient, a patient bed status (e.g., which bed in the facility is occupied by the patient), patient diagnosis, patient admit order to monitoring confirmation (if applicable) along with admit order time and recommended action (e.g., connect to monitoring, review monitoring, etc.), patient discharge order from monitoring confirmation (if applicable) along with discharge order time and recommended action (e.g., disconnect from monitoring, review monitoring, etc.), and patient monitoring status (if applicable) along with connection status and one or more thumbnails linking to a patient monitoring visualization application, a standard monitoring duration, an actual (current) monitoring duration, and a time elapsed since order.

The stratified patient list may further include, for each patient, a patient alert history (e.g., arrhythmia and limits). The patient alert history may include “high,” “medium,” “low,” and “tech” sub-headings, and in some embodiments, the patient ranking and/or patient score may be based in part on the alert history. For example, patients that having an alert history indicating a relatively high frequency of alerts (e.g., triggering events) may be ranked lower by the telemetry management system compared to other patients when the telemetry management system ranks patients according to priority for removal from telemetry monitoring. The GUI of the telemetry management system may include icons or other elements to show the alert history of patients when selected.

The stratified patient list may further include, for each patient, a summary of patient vitals (e.g., heartrate, NIBP, temperature, etc.), patient medications, patient labs, patient cardiology monitoring parameters (e.g., number and/or type of cardiology monitoring sensors coupled to the patient), and/or other patient information. The patient cardiology monitoring parameters may include, in some embodiments, one or more thumbnails linking to a cardiology monitoring visualization application (e.g., a graphical representation of cardiology monitoring sensor output). As described above, for each patient, the information of the patient may be displayed in a virtual card of the GUI of the telemetry management system. The card may include, for example, the patient bed status, patient diagnosis, patient category (as determined by the telemetry management system and described above), telemetry monitoring order status (e.g., admit order or discharge order and associated time, alerts, etc.), patient vitals, alert history summary, patient medications, patient labs, patient cardiology monitoring parameters, and/or other patient information.

The technical effect of forming the stratified patient list based on the score of each patient and displaying the stratified patient list at the display device is to quickly identify low risk patients that may be removed from telemetry monitoring based on patient information acquired by the telemetry management system from multiple different sources.

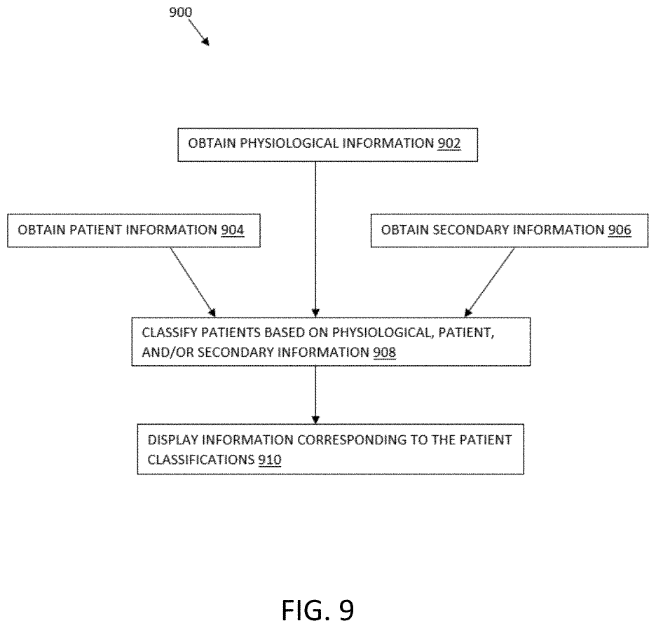

FIG. 9 is a flowchart of a process 900 of telemetry monitoring management according to an example. The telemetry processing process 900 may be performed by the telemetry management system 100.

At operation 902, physiological information may be obtained by the telemetry monitoring system 100. The physiological information may be obtained from one or more telemetry monitoring sensors 102 that may each measure multiple, different physiological parameters. For example, the physiological information may be obtained from the following non-limiting list of sensors: electrocardiogram (ECG) sensors 104, peripheral capillary oxygen saturation (SpO2) sensors 106, respiration rate (RR) sensors 103, non-invasive blood pressure (NIBP) sensors 105, and temperature sensors 101. The data provided by the telemetry monitoring sensors 102 may be normalized in real time or near real time for further processing. Normalizing the data may include filtering and/or calibrating the data. Near real time is intended to include processing and communication delays, as well as other delays caused by sending, receiving, and processing information.

At operation 904, patient information may be obtained by the telemetry monitoring system. Patient information may include information specific to the patient that is not received from a telemetry monitoring sensor 102 associated with the patient. The patient information may include, but is not limited to, information from the cardiology management system 150, the computerized physician order entry (CPOE) system 107, the electronic medical record (EMR) database 108, the laboratory information system 110, and the pharmacy information system 109. For example, patient information may include, but is not limited to, doctor's orders for the patient, prescription drug information corresponding to the patient, previous medical information, an amount of time the patient has been connected to a specific monitor, and/or laboratory results corresponding to the patient.

At operation 906, secondary information may be obtained. The secondary information may include monitoring protocols and standards that are not patient specific. That is, the secondary information may include recommended standards and protocols. The standards and protocols may be specific to certain groups of patients. For example, different standards and protocols may be used for patients of different age groups or patients having different underlying conditions. For example, a protocol may indicate an amount of time that a patient with a specific condition should be monitored. The secondary information may include other information used in monitoring that is not patient specific, such as rules or guidelines of a facility using the telemetry monitoring system 100.

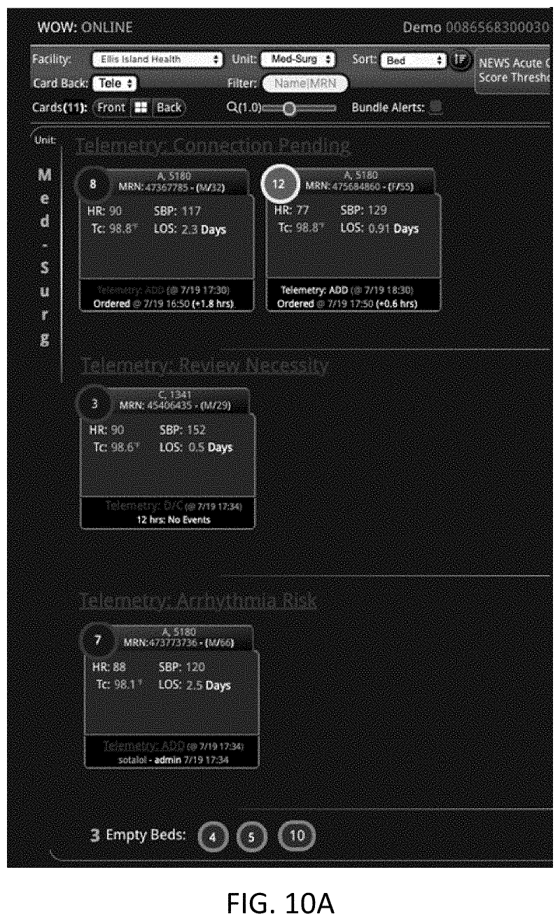

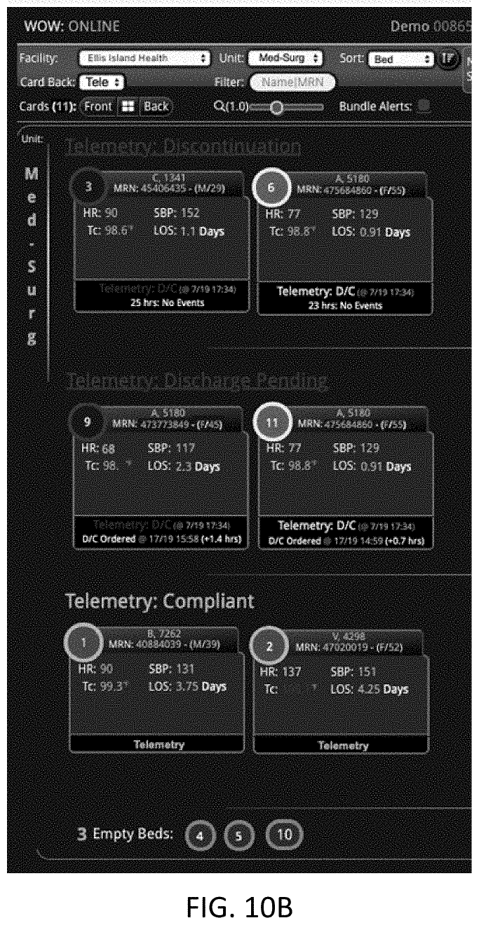

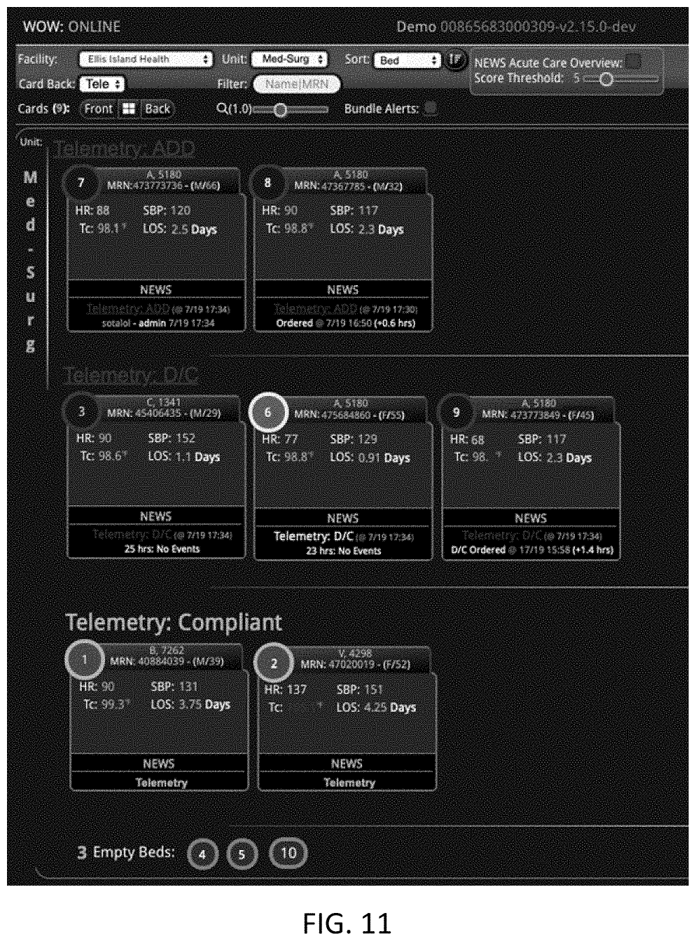

At operation 908, patients may be classified based on the patient information, the physiological information, and/or the secondary information. Each patient may be classified as an addition, a discontinuation, or compliant. According to an example, the additions may be further categorized into connection pending and condition risk, and the discontinuations may be further classified into review necessary, discontinuation, and discharge pending. At operation 910, as discussed below and shown in FIGS. 10A, 10B, and 11, information corresponding to the patient classifications may be displayed on the display device 126.

FIGS. 10A and 10B shows an example of an output of display device 126 of the telemetry management system 100. FIG. 11 shows another example of an output of display device 126 of the telemetry management system 100. For the example shown in FIGS. 10A, 10B, and 11, the patients are being monitored by patient monitoring devices which provide data corresponding to heart rate (HR), SBP, body temperature (Tc), and LOS of the patients. In this example, the telemetry monitoring sensors 102 measure ECG and SP02. As such, the telemetry monitoring sensors 102 discussed in this example will provide data in addition to the monitoring data shown in FIGS. 10A, 10B, and 11. However, the examples on FIGS. 10A, 10B, and 11, as well as the below discussion, are not intended to be limiting, but are rather provided for exemplary purposes.

According to an example, the patient information may be monitored for orders which indicate that a patient is to be connected to a telemetry monitoring. If there is no connection with a telemetry monitoring sensors 102 associated with the patient (i.e., no physiological information is being received for the patient) the patient may be classified as a “connection pending” because they are waiting to be connected to a telemetry monitoring sensor 102. As shown in the upper row of FIG. 10A, when a connection is pending, a time at which the order was placed (or input into the system) is displayed. Additionally, an amount of time that has passed since the order was place may be displayed in a window corresponding to the patient.

Each patient window may be color coded based on the severity of the monitoring event. For example, the connection pending patient on the right side may be color coded yellow because 0.6 hours has passed since the connection order was placed while the connecting pending patient on the left may be color coded red because 1.8 hours has passed since the connection order was placed. The colors may be determined based on a severity score indicating the amount of time that has passed since the monitoring event (e.g., order was placed). For example, patients having a time from 0-15 minutes may be added to the connection pending category without a color code, patients having a time from 16-59 minutes may be color coded yellow, and patients having a time over 59 minutes may be color coded red. The severity thresholds defining the example timeframes, and the amount of color coding categories in the above example are not limiting, and may be varied. For example, the severity thresholds may be varied based on factors obtained from the patient data, the physiological data, and/or the secondary data.

Each time a severity score passes a severity threshold, an action may be performed. For example, when a severity threshold is passed, one or more notifications may be sent, an alarm may be triggered, patient locations on a user interface may be rearranged, and/or instruction may be sent out to control an external device. A different action may be performed based on the severity of the threshold. For example, at a lowest threshold, a notification may be sent to a monitor or member of a care team; at a mid-threshold, an alarm me be triggered, and at a highest threshold, an instruction may be sent to control an external device (e.g., turn off a sensor that is malfunctioning, adjust parameters of a medical device connected to the patient). Further, the locations of the patient windows on the user interface may be rearranged to position the patients with the highest severity scores at the most accessible and noticeable locations of the user interface.

The above discussion of controlling a display and performing actions based on monitoring events passing threshold may be applied to any patient that is not classified as compliant. By performing these operations based on severity of a monitoring event, critical information corresponding to a group of monitored patients may be more readily distinguishable and accessible by staff monitoring the patients.

As shown in the second row of FIG. 10B, in a scenario when a patient is connected to a telemetry monitoring sensor 102, but standards or protocols of the secondary data dictate that the patient does not require monitoring (e.g., the patient's condition or symptoms do not require monitoring), a patient may be classified as “review necessary”. Since facilities have a finite amount of telemetry monitoring sensors 102, scenarios may arise where there is a shortage of monitoring sensors. Further, additional monitors may also overwhelm monitoring staff or waste resources. As such, reviewing patients who are connected to monitors when it is not necessary may result in sensor disconnections that free up resources.

In the case of a “review necessary” classification, connection to the telemetry monitoring sensor 102 may be a monitoring event. As such, an amount of time that has passed since the telemetry monitoring sensor 102 was connected to the patient may be compared to one or more thresholds as discussed above.

As shown in the third row of FIG. 10A, in a scenario when a patient is not connected to a telemetry monitoring sensor 102, but standards or protocols of the secondary data dictate that the patient does require monitoring (e.g., the patient's condition or symptoms do require monitoring), a patient may be classified as “condition risk”. For example, as shown in the bottom row of FIG. 10A, the patient corresponding to the windows has been determined to be an arrythmia risk but is not connected to a telemetry monitoring sensor. By indicating to one or more members of the care team that this patient should be connected to telemetry monitoring equipment, a higher standard of care can be provided.

In the case of a “condition risk” classification, a determination that the patient should be connected to the telemetry monitoring sensor 102 may be a monitoring event. As such, an amount of time that has passed since the determination that the patient should be connected to the telemetry monitoring sensor 102 may be compared to one or more thresholds as discussed above.

The standards and protocols may indicate an amount of time that a patient should be monitored for a specific condition. The monitoring management system 100 may determine that a telemetry monitoring sensor 102 should be removed from the patient based on patient information indicating an amount of time the patient has been connected to the telemetry monitoring sensor 102 passing a threshold defined by the secondary data. For example, as shown in the first row of FIG. 10B, if a patient is suffering from a condition in which standards and/or protocol dictate 24 hours of monitoring, and the patient data indicates that the patient has been connected to a telemetry monitoring sensor 102 for greater than 24 hours, the patient may be classified as a “discontinuation” which indicates that the patient should be removed from monitoring.

In a case of a “discontinuation” classification, the amount of time of the monitoring exceeding the amount of time indicated by the standard and/or protocol may be a monitoring event. As such, the exceeded amount of time may be compared to one or more thresholds as discussed above.

As shown in the second row of FIG. 10B, when a monitoring discharge order has been input for a patient and physiological data from the telemetry monitoring sensor 102 is still being received for the patient (e.g., patient has not been disconnected from the telemetry monitoring sensor 102), the patient may be classified as “discharge pending.” In a case of a “discharge pending” classification, the input of the monitoring discharge order may be the monitoring event. As such, an amount of time that has passed since the monitoring discharge order was input may be compared to one or more thresholds as discussed above. As shown in FIG. 10B, the display device may display the amount of time that has passed since the discharge order in a window associated with the patient.

As shown in the third row of FIG. 10B, a patient may be classified as compliant based on the patient not falling into one of the above categories (connection pending, review necessary, condition risk, discontinuation, discharge pending). For example, if physiological information is being received from a telemetry monitoring sensor 102 corresponding to a patient, an amount of time that the patient has been monitored does not exceed a standard and/or protocol of the secondary data, the patient has a condition that requires monitoring, and a discharge order has not been input for the patient, the patient may be classified as compliant.

By concurrently processing, analyzing, and classifying the patient information, the secondary information, and the real time or near real time physiological information, the user interface 128 is improved by rearranging the patients that are most relevant to an operator and most likely to be selected by the operator to a location at which they can be efficiently accessed by a user. That is, the system for telemetry monitoring management rearranges the interface based on the classifications and information corresponding to the classifications to provide an improved user interface. For example, as shown in FIGS. 10A and 10B, the patient windows are arranged into defined categories with patients having higher severity scores arranged to the left.

The classifications and arrangement of patients on the user interface may be based on the stratified list. For example, patients higher on the stratified list may arranged in a more noticeable or convenient location on the user interface.

FIG. 11 shows an embodiment in which the monitored patients are classified into 3 categories: addition (first row), discontinuation (middle row), and compliant (bottom row). The classifications may be performed as discussed above where “connection pending” and “condition risk” are classified as an “addition,” and “review necessary,” “discontinuation,” and “discharge pending” are classified as a “discontinuation”.

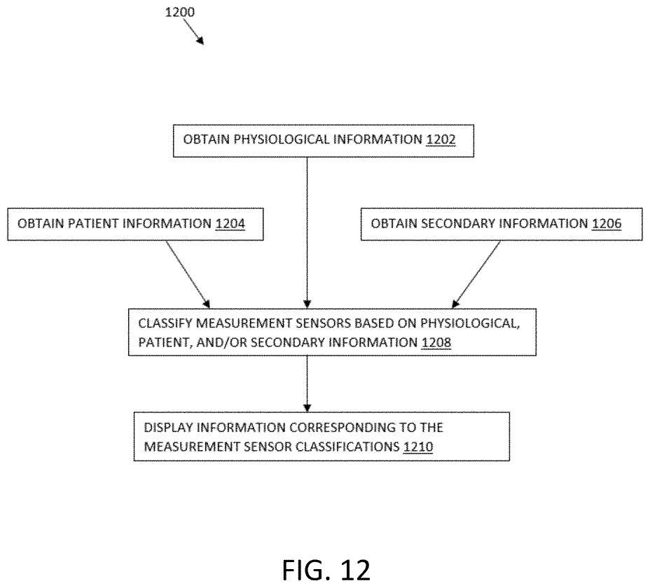

FIG. 12 is a flowchart of another process 1200 of telemetry monitoring management according to an example. The telemetry processing process 1200 may be performed by the telemetry management system 100.

Telemetry monitoring process 1200 is similar to telemetry process 900, with the difference being that the classifications are performed for each measurement sensor in operation 1208. As discussed above, each telemetry monitoring sensor 102 may be connected to multiple measurement sensors that each measure a different physiological parameter of a patient. According to the telemetry monitoring process 1200, a classification is performed for each sensor and the user interface may be controlled based on these classifications. For example, when a patient is connected to a telemetry monitoring sensor 102, and a connection order is input for a measurement sensor that is not connected to the telemetry monitoring sensor 102, information pertaining to the specific measurement sensor may be readily available to a user through the user interface 128.

According to an example, a system for telemetry monitoring may include: a plurality of telemetry monitoring sensors, each telemetry monitoring sensor may include: a battery; a radio antenna configured to communicate over a wireless network; at least one sensor configured to measure a physiological parameter; a memory configured to store instructions; and a controller configured to execute the instructions to control the radio antenna to send electronic signals over the wireless network that include real time or near real time machine data obtained from the at least one sensor, the real time or near real time machine data including physiological information. The system for telemetry monitoring may also include a telemetry management system which may include: a display device; a communication interface configured to communicate over the wireless network; a memory configured to store instructions; at least one processor configured to execute the instructions to: obtain patient information for a plurality of patients; receive, through the communication interface, the physiological information from the plurality of telemetry monitoring sensors, the physiological information respectively corresponding to the plurality of patients; obtain secondary information including patient monitoring standards and protocols; classify each patient of the plurality of patients based on one or more of the patient information, the physiological information, and the secondary information; and control the display device to display information corresponding to the classifications the plurality of patients, and wherein the classifications include addition, discontinuation, and compliant.

According to an example, the at least one sensor may include one or more of an ECG sensor, a blood oxygen saturation sensor, a blood pressure sensor, a respiration rate sensor, a heart rate sensor, and a temperature sensor.

According to an example, at least one processor may be further configured to: concurrently normalize each stream of machine data from the plurality of telemetry monitoring sensors; and continuously analyze each normalized stream of machine data from the plurality of telemetry monitoring sensors to extract the physiological parameters from the data.

According to an example, the at least one processor may be further configured to: for each patient assigned a telemetry monitoring sensor, determine a classification of the patient based on the extracted physiological parameters; and control the display device to display a change in the classification of each patient in real time or near real time.

According to an example, the system for telemetry monitoring may include a database storing the secondary data. The at least one processor may be further configured to: continuously monitor the patient data of each patient among the plurality of patients for a change in monitoring status; and based on detecting a change in monitoring status for a patient, accessing the database to obtain secondary data pertaining to the changed monitoring status.

According to an example, the at least one processor may be further configured to classify a patient as an addition based on one of: the patient information indicating telemetry monitoring has been ordered for the patient and not receiving physiological information from a telemetry monitoring sensor assigned to the patient; and a comparison between the secondary information and patient information of the patient indicating that the patient should be monitored and not receiving physiological information from a telemetry monitoring sensor assigned to the patient.

According to an example, the at least one processor may be further configured to, based on the patient being classified as the addition, control the display to display a timer indicating an amount of time that has elapsed since one of: a timepoint at which the telemetry monitoring was ordered for the patient; and a timepoint at which the comparison indicated that the patient should be monitored.

According to an example, the comparison between the secondary information and the patient information of the patient indicates that the patient should be monitored based on the patient information of the patient not conforming with the patient monitoring protocols and/or standards of the secondary information.

According to an example, the at least one processor may be further configured to classify a patient as a discontinuation based on one of: the patient information indicating that telemetry discharge has been ordered for the patient and physiological information from a telemetry monitoring sensor assigned to the patient is being received; and a comparison between the secondary information and patient information of the patient indicating that the patient monitoring should be discontinued while receiving physiological information from a telemetry monitoring sensor assigned to the patient.

According to an example, the at least one processor may be further configured to, based on the patient being classified as the discontinuation, control the display to display a timer indicating an amount of time that has elapsed since one of the telemetry discharge was ordered and the comparison indicated that the patient monitoring should be discontinued.

According to an example, the comparison between the secondary information and patient information of the patient indicates that the patient monitoring should be discontinued based on one of: the secondary information indicating that a medical condition specified by the patient information of the patient does not require telemetry monitoring; and a time of telemetry monitoring specified by the patient information of the patient being greater than a monitoring threshold defined by the patient monitoring standards and protocols of the secondary information.

According to an example the at least one processor may be further configured to, for each patient classified as an addition or discontinuation: determine a severity scope for the patient; compare the severity score to one or more predefined thresholds; and control the display device to display a severity indicator corresponding to the patient based on the most sever threshold passed.

According to an example, the at least one processor may be further configured to, for each patient classified as an addition or discontinuation: determine a severity scope for the patient; compare the severity score to one or more predefined thresholds; and based on the severity score passing one of the one or more predefined thresholds, send a notification corresponding to the passed threshold or trigger an alarm corresponding to the passed threshold.

According to an example, the at least one processor may be further configured to, for each patient classified as an addition or discontinuation: determine a severity scope for the patient; compare the severity score to one or more predefined thresholds; and adjust, based on the severity score passing one of the one or more predefined thresholds, a parameter of one of the at least one sensors.

According to an example, the at least one processor may be further configured to, for a patient among the plurality of patients: determine, based on patient information of the patient and the secondary information, a best device for monitoring the patient; and provide a best device recommendation based on a telemetry monitoring device associated with the patient being different than the determined best device.

In cases where a patient has access to multiple monitoring devices, the telemetry management system may recommend a best device for the patient. For example different monitoring device may have different physiological sensors, different capabilities (e.g. movable, usable with imaging equipment, ect), and different strengths (e.g. longer battery life, longer wireless connection range). Accordingly, the telemetry management system may look at the patient information and recommend a best patient monitor based in this information. For example, if a patient requires ECG and SPO2 monitoring, and a first device provides ECG and SPO2 sensors, while a second device provides respiration rate and SPO2 sensors, the telemetry management system may recommend the first device.

According to an example, the telemetry management system may determine a best device or if no device is the best option. For example, if no devices measure the parameters that need to be monitored, the system may recommend no use of a device.

According to an example, the at least one processor may be further configured to: determine an amount of available telemetry monitoring sensors that are not in use; obtain a pending value by adding an amount of telemetry sensors classified as a discontinuation to an amount of telemetry sensors classified as an addition; and obtain a potential amount of available telemetry sensors by adding the pending value to the amount of available telemetry sensors.

The telemetry monitoring system may monitor a current amount of available/unconnected telemetry monitoring sensors. However, an amount of potentially available telemetry monitoring sensors, based on the pending additions and discontinuations, may be usefully information for a monitoring staff. As such, the amount of potential available telemetry monitoring sensors may be determined by subtracting the patients classified as additions from the patients classified as discontinuations, and then adding the difference to the current amount of available/unconnected telemetry monitoring sensors. This forecasting process may be used for other types of sensors and resources.

According to an example, the processor may be further configured to: classify each sensor, among the at least one sensor, assigned to a patient among the plurality of patients based on one or more of the patient information, the physiological information, and the secondary information; and control the display device to display information corresponding to the sensor classifications.

According to an example, the at least one processor may be further configured to: classify a patient as an addition based on one of: the patient information indicating telemetry monitoring has been ordered for the patient and not receiving physiological information from a telemetry monitoring sensor assigned to the patient; and a comparison between the secondary information and patient information of the patient indicating that the patient should be monitored and not receiving physiological information from a telemetry monitoring sensor assigned to the patient. The at least one processor may be further configured to classify a patient as a discontinuation based on one of: the patient information indicating telemetry discharge has been ordered for the patient and physiological information from a telemetry monitoring sensor assigned to the patient is being received; and a comparison between the secondary information and patient information of the patient indicating that the patient monitoring should be discontinued while receiving physiological information from a telemetry monitoring sensor assigned to the patient.

According to an example, a system for telemetry monitoring may include: a plurality of telemetry monitoring sensors, each telemetry monitoring sensor comprising: a measurement sensor configured to measure a physiological parameter; a memory configured to store instructions; and a controller configured to execute the instructions to control to send real time or near real time machine data obtained from the measurement sensor, the real time or near real time machine data including physiological information; and a telemetry management system including: a display device; a communication interface configured to communicate over the wireless network; a memory configured to store instructions; at least one processor configured to execute the instructions to: obtain patient information for a plurality of patients, wherein the patient information comprises sensor information for each patient among the plurality of patients; receive, through the communication interface, the physiological information from the plurality of telemetry monitoring sensors, the physiological information respectively corresponding to the plurality of patients; obtain secondary information including patient monitoring standards and protocols; classify each sensor of the sensor information based on one or more of the patient information, the physiological information, and the secondary information; and control the display device to display information corresponding to the classifications the sensors, and wherein the classifications include addition, discontinuation, and compliant.

According to an example, a telemetry management system may include: a communication interface configured to communicate over a network; a memory configured to store instructions; at least one processor configured to execute the instructions to: obtain patient information for a plurality of patients; receive, through the communication interface, the physiological information from the plurality of telemetry monitoring sensors, the physiological information respectively corresponding to the plurality of patients; obtain secondary information including patient monitoring standards and protocols; classify each patient of the plurality of patients based on one or more of the patient information, the physiological information, and the secondary information; and output, through the communication interface, information corresponding to the classifications of the plurality of patients, and wherein the classifications include addition, discontinuation, and compliant.

According to an example, when a severity indicator passes a threshold, notifications may be sent to an alarm management/notification system for further distribution. The system may distribute the notifications over a network to one or more members of a care team based on a distribution list. For example, the notifications may be distributed to one or more specific members of the care team based on information indicating that the member of the care team is caring for a specific patient at a specific time. The system may also determine to whom to send the notification based on information corresponding to the threshold being passed. For example, a lower threshold may cause the notification to be sent to a caregiver while a high threshold may cause the notification to be sent to multiple members of the care team.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A system for telemetry monitoring comprising:
a plurality of telemetry monitoring sensors, each telemetry monitoring sensor comprising:
a battery;
a radio antenna configured to communicate over a wireless network;
at least one sensor configured to measure a physiological parameter;
a memory configured to store instructions; and
a controller configured to execute the instructions to control the radio antenna to send electronic signals over the wireless network that include real time or near real time machine data obtained from the at least one sensor, the real time or near real time machine data including physiological information; and
a telemetry management system comprising:
a display device;
a communication interface configured to communicate over the wireless network;
a memory configured to store instructions; and
at least one processor configured to execute the instructions to:
obtain patient information for a plurality of patients;
receive, through the communication interface, the physiological information from the plurality of telemetry monitoring sensors, the physiological information respectively corresponding to the plurality of patients;
obtain secondary information including patient monitoring standards and protocols;
classify each patient of the plurality of patients based on one or more of the patient information, the physiological information, and the secondary information; and
control the display device to display information corresponding to the classifications the plurality of patients, and
wherein the classifications include addition, discontinuation, and compliant.

2. The system of claim 1, wherein the at least one sensor comprises one or more of an ECG sensor, a blood oxygen saturation sensor, a blood pressure sensor, a respiration rate sensor, a heart rate sensor, and a temperature sensor.

3. The system of claim 1, wherein at least one processor is further configured to:
concurrently normalize each stream of machine data from the plurality of telemetry monitoring sensors; and
continuously analyze each normalized stream of machine data from the plurality of telemetry monitoring sensors to extract the physiological parameters from the data.

4. The system of claim 3, wherein the at least one processor is further configured to:
for each patient assigned a telemetry monitoring sensor, determine a classification of the patient based on the extracted physiological parameters; and
control the display device to display a change in the classification of each patient in real time or near real time.

5. The system of claim 1, further comprising a database storing the secondary data, wherein the at least one processor is further configured to:
continuously monitor the patient data of each patient among the plurality of patients for a change in monitoring status; and
based on detecting a change in monitoring status for a patient, accessing the database to obtain secondary data pertaining to the changed monitoring status.

6. The system of claim 1, wherein the at least one processor is further configured to classify a patient as an addition based on one of:
the patient information indicating telemetry monitoring has been ordered for the patient and not receiving physiological information from a telemetry monitoring sensor assigned to the patient; and
a comparison between the secondary information and patient information of the patient indicating that the patient should be monitored and not receiving physiological information from a telemetry monitoring sensor assigned to the patient.

7. The system of claim 6, wherein the at least one processor is further configured to, based on the patient being classified as the addition, control the display to display a timer indicating an amount of time that has elapsed since one of:
a timepoint at which the telemetry monitoring was ordered for the patient; and
a timepoint at which the comparison indicated that the patient should be monitored.

8. The system of claim 6, wherein the comparison between the secondary information and the patient information of the patient indicates that the patient should be monitored based on the patient information of the patient conforming with the protocols and/or standards of the secondary information.

9. The system of claim 1, wherein the at least one processor is further configured to classify a patient as a discontinuation based on one of:
the patient information indicating that telemetry discharge has been ordered for the patient and physiological information from a telemetry monitoring sensor assigned to the patient is being received; and
a comparison between the secondary information and patient information of the patient indicating that the patient monitoring should be discontinued while receiving physiological information from a telemetry monitoring sensor assigned to the patient.

10. The system of claim 9, wherein the at least one processor is further configured to, based on the patient being classified as the discontinuation, control the display to display a timer indicating an amount of time that has elapsed since one of:
a timepoint at which the telemetry discharge was ordered; and
a timepoint at which the comparison indicated that the patient monitoring should be discontinued.

11. The system of claim 9, wherein the comparison between the secondary information and patient information of the patient indicates that the patient monitoring should be discontinued based on one of:
the secondary information indicating that a medical condition specified by the patient information of the patient does not require telemetry monitoring; and
a time of telemetry monitoring specified by the patient information of the patient being greater than a monitoring threshold defined by the patient monitoring standards and protocols of the secondary information.

12. The system of claim 1, wherein the at least one processor is further configured to, for each patient classified as an addition or discontinuation:
determine a severity scope for the patient;
compare the severity score to one or more predefined thresholds; and
control the display device to display a severity indicator corresponding to the patient based on the most severe threshold passed.

13. The system of claim 1, wherein the at least one processor is further configured to, for each patient classified as an addition or discontinuation:
determine a severity scope for the patient;
compare the severity score to one or more predefined thresholds; and
based on the severity score passing one of the one or more predefined thresholds, send a notification corresponding to the passed threshold or trigger an alarm corresponding to the passed threshold.

14. The system of claim 1, wherein the at least one processor is further configured to, for each patient classified as an addition or discontinuation:
determine a severity scope for the patient;
compare the severity score to one or more predefined thresholds; and
adjust, based on the severity score passing one of the one or more predefined thresholds, a parameter of one of the at least one sensors.

15. The system of claim 1, wherein the at least one processor is further configured to, for a patient among the plurality of patients:
determine, based on patient information of the patient and the secondary information, a best device for monitoring the patient; and
provide a best device recommendation based on a telemetry monitoring device associated with the patient being different than the determined best device.

16. The system of claim 1, wherein the at least one processor is further configured to:
determine an amount of available telemetry monitoring sensors that are not in use;
obtain a pending value by adding an amount of telemetry sensors classified as a discontinuation to an amount of telemetry sensors classified as an addition; and
obtain a potential amount of available telemetry sensors by adding the pending value to the amount of available telemetry sensors.

17. The system of claim 1, wherein the processor is further configured to:
classify each sensor, among the at least one sensor, assigned to a patient among the plurality of patients based on one or more of the patient information, the physiological information, and the secondary information; and
control the display device to display information corresponding to the sensor classifications.

18. The system of claim 17, wherein the at least one processor is further configured to: classify a patient as an addition based on one of:
the patient information indicating telemetry monitoring has been ordered for the patient and not receiving physiological information from a telemetry monitoring sensor assigned to the patient; and
a comparison between the secondary information and patient information of the patient indicating that the patient should be monitored and not receiving physiological information from a telemetry monitoring sensor assigned to the patient, and wherein the at least one processor is further configured to classify a patient as a discontinuation based on one of:
the patient information indicating telemetry discharge has been ordered for the patient and physiological information from a telemetry monitoring sensor assigned to the patient is being received; and
a comparison between the secondary information and patient information of the patient indicating that the patient monitoring should be discontinued while receiving physiological information from a telemetry monitoring sensor assigned to the patient.

19. A system for telemetry monitoring comprising:
a plurality of telemetry monitoring sensors, each telemetry monitoring sensor comprising:
a measurement sensor configured to measure a physiological parameter;
a memory configured to store instructions; and
a controller configured to execute the instructions to control to send real time or near real time machine data obtained from the measurement sensor, the real time or near real time machine data including physiological information; and
a telemetry management system comprising:
a display device;
a communication interface configured to communicate over the wireless network;
a memory configured to store instructions;
at least one processor configured to execute the instructions to:
obtain patient information for a plurality of patients, wherein the patient information comprises sensor information for each patient among the plurality of patients;
receive, through the communication interface, the physiological information from the plurality of telemetry monitoring sensors, the physiological information respectively corresponding to the plurality of patients;
obtain secondary information including patient monitoring standards and protocols;
classify each sensor of the sensor information based on one or more of the patient information, the physiological information, and the secondary information; and
control the display device to display information corresponding to the classifications the sensors, and
wherein the classifications include addition, discontinuation, and compliant.

20. A telemetry management system comprising:
a communication interface configured to communicate over a network;
a memory configured to store instructions;
at least one processor configured to execute the instructions to:
obtain patient information for a plurality of patients;
receive, through the communication interface, the physiological information from the plurality of telemetry monitoring sensors, the physiological information respectively corresponding to the plurality of patients;
obtain secondary information including patient monitoring standards and protocols;
classify each patient of the plurality of patients based on one or more of the patient information, the physiological information, and the secondary information; and
output, through the communication interface, information corresponding;
to the classifications of the plurality of patients, and wherein the classifications include addition, discontinuation, and compliant.
